SEQUENCE LISTING (14 sequences)

SEQ ID NO: 1 - protein, 215 aa, Ostertagia Ostertagi
GFCCPADLNQTDEARKIFLDFHNQVRRDIAGASPLLNLTGAVQMRNVLGPAKNMYRMDWDCNLEAKAKAMIWPCTTPLPIDTSIPQNLAQWLLFQNSQENEVLTQTPWSWVTASLRNLQPDTEANIYNWQIRPLSNIANWQNLKVGCAHKVCKFPTGTNMVVSCAYGGEVLQDNEVVWDKGPTCMCNAYPNSFCCNNLCDTIAAATLRNQPCKST

SEQ ID NO: 2 - protein, 422 aa, Cooperia oncophora
DEIRKVFLDKHNEYRSLVAKGQALNPQFGGSTPKAARMLKARYDCDVEEDMTKWAQAQCTYAPFKSSKRYGRNTWGMGVPNYNKTAAAESSVYDWFFELRRYGVPQDNVYTRDVDYSAYHYAQMVWQDSYKIGCVVAWCPSMTWVACGYSPAGDNIGSLIYELGEPCTKNEDCKCTDCTCSEGEALCIPPGGPKPATTASTTTKTTTTTKPTTTTTEPSTTTAKPTTTFDRAAWEESVKRPVARCTLDNGMTDEARQVFLDKHNEYRQLVARGEAKNKTGLAPPAARMLQMRYDCDLEAHVMEHVKQCKGGHSSFDVLKGRGQNIWAITVPNLDKADAANRSVHDWYIELTKYGITADNKISMDNAANTGHYSQVVWQKSNRLGCAAVSCPEQGKLFVGCEYLPGGNTLHHLIYDIGEPCKR

SEQ ID NO: 3 - protein, 422 aa, Cooperia oncophora
DEIRKVFLDKHNEYRSLVAKGQAPNPQFGGSTPKAARMLKAMYDCDVEEDMTKWAQAQCTYAPFKSSKHYGRNTWGMGVPNYNKTAAAESSVYDWFSELRRYGVPQDNVYTRDVDYSAYQYAQMVWQDSYKIGCVVAWCPSVTWVACGYSPAGDNIGSLIYELGEPCTKNEDCKCTDCTCSEGEALCIPPGGPKPATTASTTTKTTTTTKPTTTTTKLSTTTAKPTTTFDRAAWEESVKRPVARCTLDNGMTDEARQVFLDKHNEYRQLVARGEAKNKTGLAPPAARMLQMRYDCDLEAHVMEHVKQCKGGHSSFDVLKGRGQNIWAITVPNLDKADAANRSVHDWYIELTKYGITADNKISMDNAANTGHYSQVVWQKSNRLGCAAVSCPEQGKLFVGCEYLPGGNTLHHLIYDIGEPCKR

SEQ ID NO: 4 - protein, 422 aa, Cooperia oncophora
DEIRKVFLDKHNEYRSLVAKGQAPNPQFGGSTPKAARMLKARYDCDVEEDMTKWAQAQCTYAPFKSSKRYGRNTWGMGVPNYNKIAAAESSVDDWFFELRRYGVPQDNVYTRDVDYSAYHYAQMVWQDSYKIGCVVAWCPSMTWVACGYSPAGDNIGSLIYELGEPCTKNEDCKCTDCTCSEGEALCIPPGEPKPATTASTTTKTTTTTEPTTTTTEPSTTTAKPTTTFDRAAWEESVKRPVARCTLDNGMTDEARQVFLDKHNEYRQLVARGEAKNKTGLAPPAARMLQMRYDCDLEAHVMEHVKQCKGGHSSFDVLKGRGQNIWAITVPNLDKADAANRSVHDWYIELTKYGITADNKISMDNAANTGHYSQVVWQKSNRLGCAAVSCPEQGKLFVGCEYLPGGNTLHHLIYDIGEPCKR

SEQ ID NO: 5 - protein, 215 aa, Cooperia oncophora
GFCCPADLDQTDEARKILLNFHNEVRRDVSSASPLLNLTGAVLMRNVLGPAKNMYKMDWDCNLEKKALEMISPCTVPLPIDTSIPQNLAQWLLYRKMEETEVLEKAPWSWVIASLRNLKNDTEADLYNWKIRTISNILNWRNTKVGCAHKVCQFPTGTNMVISCAYGGDKLENNEVVWQKGPTCECNAYPDSYCCNNLCDTKAAAALREEPCKSN

SEQ ID NO: 6 - protein, 9 aa, Cooperia oncophora
LCSLDNGMT

SEQ ID NO: 7 - protein, 22 aa, Cooperia oncophora
DEDCKCSSCRCSTQLSMCINPN

SEQ ID NO: 8 - protein, 42 aa, Ostertagia Ostertagi
GFCCPADLNQTDEARKIFLDFHNQVRRDIAGASPLLNLTGAV

SEQ ID NO: 9 - protein, 23 aa, Ostertagia Ostertagi
GFCCPADLNQTDEARKIFLDFHN

SEQ ID NO: 10 - DNA, 1266 bp, Cooperia oncophora
  source 1..1266: mol_type = other DNA
gatgagatcagaaaagtcttccttgacaagcacaatgagtaccggtcactcgttgctaaaggacaggctctgaatccacagtttggcgggtctactccaaaggccgctagaatgctcaaagcgaggtacgattgcgatgttgaagaagacatgacgaagtgggctcaagcgcagtgcacgtacgcaccattcaaaagtagcaaacgttacggccggaacacatggggcatgggtgtccctaactacaacaagacagcagctgcagaatcgagtgtttacgactggttcttcgaactacggcgctatggtgttcctcaagataacgtgtatacaagagatgttgactacagtgcttatcattacgctcagatggtttggcaagatagttacaaaattggatgtgtcgtggcatggtgtccaagcatgacctgggtagcatgcggatacagtccagcaggagataatatcggatccctaatttacgagcttggagaaccgtgtacaaagaatgaagactgtaaatgcaccgactgcacatgtagtgaaggagaagctctttgtatacctcctggaggaccgaaacccgctaccactgcaagcaccacgaccaagacaacgaccactacaaagcctacgacgacgacgacggaaccttcgaccactacggcgaagccaacgacgaccttcgatagagctgcgtgggaggagtcggtcaagaggccagtagcgcgttgcactcttgacaacggaatgacagacgaggccaggcaggttttccttgacaagcacaacgagtaccggcaactagttgcaagaggagaagctaaaaacaagacaggattggctccgccggcagctagaatgctacaaatgaggtacgattgcgaccttgaggcacatgttatggagcacgttaaacagtgtaaaggcggacattcatcatttgatgtgcttaaaggtagggggcagaacatatgggccataactgtccctaacttggacaaggctgatgctgcaaaccggagtgtccatgactggtacatcgaattaacgaaatatggtataaccgcagataacaagatatcaatggacaatgctgcaaacactggtcattactcgcaggtagtttggcaaaagtcgaacagacttggatgtgcagcggtgtcctgtccagaacaaggaaaactctttgtaggttgcgaatatttaccaggagggaacacacttcaccatctgatttacgatatcggagagccatgcaaacgg

SEQ ID NO: 11 - DNA, 1266 bp, Cooperia oncophora
  source 1..1266: mol_type = other DNA
gatgagatcaggaaagtcttccttgacaagcacaatgagtatcggtcactcgttgctaaaggacaggctccgaatccacagtttggcggatctactccaaaggccgctagaatgctcaaagcgatgtacgattgcgatgttgaagaagacatgacgaagtgggctcaagcgcagtgcacgtacgcaccattcaaaagtagcaaacattacggccggaacacatggggcatgggtgtccctaactacaacaagacagcagctgcagaatcgagtgtttacgactggttctccgaactacggcgctatggtgttcctcaagataacgtgtatacaagagatgttgactacagtgcttatcaatacgctcagatggtttggcaagacagttacaaaattggatgtgtcgtggcatggtgtccaagcgtgacctgggtagcgtgcggatacagtccagcaggagataatatcggatccctaatttacgagcttggagaaccgtgtacgaagaatgaagactgtaaatgcaccgactgcacatgtagtgaaggagaagctctttgtatacctcctggaggaccaaaacccgctaccactgcaagcaccacaaccaagacaacgaccactacaaagcctacgacgacgacgacgaaactttcgaccactacggcgaagccaacgacgaccttcgatagagctgcgtgggaggagtcggtcaagaggccagtagcgcgttgcactcttgacaacggaatgacagacgaggccaggcaggttttccttgacaagcacaacgagtaccgacaactagttgcaagaggagaagctaaaaacaagacaggattggctccgccggcagctagaatgctacaaatgaggtacgattgcgaccttgaggcacatgttatggagcacgttaaacaatgtaaaggcggacattcatcatttgatgtgcttaaaggtagggggcagaacatatgggccataactgtccctaacttggacaaggctgatgctgcaaaccggagtgtccatgactggtacatcgaattaacgaaatatggtataactgcagataacaagatatcaatggacaatgctgcaaacactggtcattactcgcaggtagtttggcaaaagtcgaacagacttggatgtgcagcggtgtcctgtccagaacaaggaaaactctttgtaggttgcgaatatttaccaggagggaacacacttcaccatctgatttacgatatcggagagccatgcaaacgg

SEQ ID NO: 12 - DNA, 1266 bp, Cooperia oncophora
  source 1..1266: mol_type = other DNA
gatgagatcagaaaagtcttccttgacaagcacaatgagtatcggtcactcgttgctaaaggacaggctccgaatccacagtttggcgggtctactccaaaggccgctagaatgctcaaagcgaggtacgattgcgatgttgaagaagacatgacgaagtgggctcaagcgcagtgcacgtacgcaccattcaaaagtagcaaacgttacggccggaacacatggggaatgggtgtccctaactacaacaagatagcagctgcagaatcgagtgttgacgactggttcttcgaactacggcgctatggtgttcctcaagataacgtgtatacaagagatgttgactacagtgcttatcattacgctcagatggtttggcaagacagttacaaaattggatgtgtcgtggcatggtgtccaagcatgacctgggtagcgtgcggatacagtccagcaggagataatatcggatccctaatttacgagcttggagaaccgtgtacaaagaatgaagactgtaaatgcaccgactgcacatgtagtgaaggagaagctctttgtatacctcctggagaaccgaaacccgctaccactgcaagcaccacgaccaagacaacgaccactacagagcctacgacaacgacgacggaaccgtcgaccactacggcgaagccaacgacgaccttcgatagagctgcgtgggaggagtcggtcaagaggccagtagcgcgttgcactcttgacaacggaatgacagacgaggccaggcaggtttccctcgacaagcacaacgagtaccggcaactagttgcaagaggagaagctaaaaacaagacaggattggctccgccggcagctagaatgctacaaatgaggtacgattgcgaccttgaggcacatgttatggagcacgttaaacaatgtaaaggcggacattcatcatttgatgtgcttaaaggtagggggcagaacatatgggccataactgtccctaacttggacaaggctgatgctgcaaaccggagtgtccatgactggtacatcgaattaacgaaatatggtataactgcagataacaagatatcaatggacaatgctgcaaacactggtcattactcgcaggtagtttggcaaaagtcgaacagacttggatgtgcagcggtgtcctgtccagaacaaggaaaactctttgtaggttgcgaatatttaccaggagggaacacacttcaccatctgatttacgatatcggagagccatgcaaacgg

SEQ ID NO: 13 - protein, 21 aa, Ostertagia Ostertagi
MQALIGIAALYLVLVTSNTEA

SEQ ID NO: 14 - protein, 21 aa, Teladorsagia circumcincta
MFTPIGIAVLYLALVTPHAKA

(19) United States
(12) Patent Application Publication
(10) Pub. No.: US 2026/0152530 A1
(43) Pub. Date: Jun. 4, 2026

(54) RECOMBINANT ACTIVATION-ASSOCIATED SECRETED PROTEIN

(71) Applicants: Universiteit Gent, Gent (BE); Academisch Ziekenhuis Leiden, Leiden (NL); Wageningen Universiteit, Wageningen (NL)

(72) Inventors: Peter GELDHOF, Gent (BE); Laurens ZWANENBURG, Gent (BE); Jimmy BORLOO, Gent (BE); Ruud Hendrikus Petrus WILBERS, Wageningen (NL); Cornelis Hendrik HOKKE, Leiden (NL); Angela VAN DIEPEN, Leiden (NL)

(21) Appl. No.: 19/123,963

(22) PCT Filed: Oct. 27, 2023
(86) PCT No.: PCT/EP2023/080078

§ 371 Date: Apr. 24, 2025

(30) Foreign Application Priority Data
Oct. 27, 2022 (EP) 22204069.3

(51) Int. Cl. C07K 14/435; A61K 39/00; A61P 33/10; A61P 37/04; C12N 9/10

(52) U.S. Cl. CPC C07K 14/4354; A61K 39/0003; A61P 33/10; A61P 37/04; C12N 9/1051

(57) ABSTRACT

The present invention relates to a recombinant activation-associated secreted protein (ASP) or fragment thereof, said ASP or fragment comprising an N-glycan comprising a core α1,3-fucose and/or a core α1,6-fucose (Fuc). The invention further relates to a pharmaceutical composition comprising such a recombinant ASP or fragment thereof. Additionally, the invention relates to the recombinant ASP or fragment thereof for use as a human or veterinary medicine, in particular as a vaccine, more in particular for use against parasitic nematode infections.

### FIELD OF THE INVENTION

The present invention relates to a recombinant activation-associated secreted protein (ASP) or fragment thereof which can for example be obtained from an Nicotiana spp., Pichia spp., or insect cell line expression system. The invention further relates to a pharmaceutical composition comprising such a recombinant ASP or fragment thereof. Additionally, the invention relates to the recombinant ASP or fragment thereof for use as a human or veterinary medicine, in particular as a vaccine; more in particular for use against parasitic nematode infections.

### BACKGROUND OF THE INVENTION

Control of the gastrointestinal nematode Ostertagia ostertagi harbours a substantial economic importance for livestock farms as infections hold a weighty share in production loss.

Treatment of these and similar helminth infections in cattle relies almost entirely on the application of anthelmintic drugs but increasing reports of upcoming anthelmintic resistance in Cooperia oncophora hampers future perspectives of these compounds. Also, for O. ostertagi, reports of anthelmintic resistance have been made. To overcome this obstacle, alternative and sustainable control measures, such as vaccination, have been explored in recent years.

There currently is an experimental vaccine for O. ostertagi that is based on endogenous activation-associated secreted proteins (ASP) purified from the excretory-secretory (ES) proteins released predominantly by adult worms, from here on referred to as Oo-ASP. Although the exact role of ASP is still largely unclear, it has been insinuated that it plays a role in helminth survival, propagation, host infection, and immune evasion as it represents a portion of the ES proteins. The fact that cysteine-rich secretory/antigen 5/pathogenesis-related 1 (CAP), a superfamily to which ASP belongs, are able to sequester small hydrophobic ligands such as sterols further underlines the fact that modulation of host-immune response in favor of the parasite is probable.

Under experimental conditions, intramuscular immunization of cattle with these ASP antigens significantly reduces the egg excretion and worm burden. Looking further into immunological research, it is demonstrated that vaccination with this antigen results in a significant proliferation of NK cells, IFN-γ secretion and a mixed IgG1/IgG2 antibody response compared to a negative—adjuvant-only—vaccine group.

As obtaining a large quantity of endogenous vaccine antigens is too cumbersome to fit an economical application, recombinant expression is essential for its future perspectives. Besides, an economic application of these endogenous antigens is ethically irresponsible hence the fact that the acquirement of Oo-ASP requires euthanasia of calves. Despite the protective capacity of the endogenous Oo-ASP, no significant reduction in faecal egg output and/or worm counts was obtained after immunization of calves with a Pichia pastoris recombinant (Gonzalez-Hernandez et al., 2016). It was also pointed out in this study that only animals vaccinated with the endogenous Oo-ASP showed a marked secondary antibody response in the gastrointestinal tract, in particular IgG1 and IgG2 levels. In addition, the cellular memory response induced by the endogenous Oo-ASP was notably stronger in contrast to the recombinant P. pastoris counterpart. Also, for C. oncophora, a comparative analysis of the immune responses induced by native versus recombinant versions of the ASP-based vaccine demonstrated significant levels of protection after an experimental challenge infection, whereas the recombinant vaccine did not (Gonzalez Hernandez et al. 2018). These findings indicate that the endogenous ASP triggers the (bovine) immune system in a unique manner, which, so far, we have not been able to mimic using recombinant forms of the ASP. In turn, this implies that the recombinants contain structural differences and/or substantially lack the antigenic epitopes which are present on the endogenous ASP.

In order to get a better understanding on the immunogenic capacity of ASP it is essential to determine which (primary and/or secondary) structural features, subunit(s) of the protein and/or its N-glycosylation are part of the antibody epitope and consequently the development of protective immunity.

During the research leading to this invention, the inventors compared the endogenous ASP to its recombinant counterpart to explore the biochemical differences governing protection. In addition peptide and glycan microarrays were implemented to study the interaction of antigen-specific antibodies and highly specified regions or epitopes on these antigens.

It is an object of the invention to provide a recombinant ASP which induces an immune response against parasitic nematodes and which can be produced on large scale.

It was surprisingly found that the presence of a N-glycan comprising a core α1,3-fucose and/or a core α1,6-fucose, and in particular a core α1,3-fucose, is essential for a recombinant ASP to be bound by native ASP induced IgG's. Recombinant ASP having an N-glycan comprising a core α1,3-fucose, a core α1,6-fucose, or combinations thereof, can therefore be used to induce an immune response. Further, due its ability to be produced via different expression systems such as the Nicotiana benthamiana expression system, Pichia pastoris expression system, or an insect cell line expression system, the recombinant ASP having an N-glycan comprising a α1,3-fucose and/or a α1,6-fucose can be produced on large scale.

### SUMMARY OF THE INVENTION

In a first aspect, the invention provides a recombinant activation-associated secreted protein (ASP) or fragment thereof, said ASP or fragment comprising an N-glycan comprising a core α1,3-fucose and/or a core α1,6-fucose (Fuc), in particular a core α1,3-fucose.

In a particular embodiment of the invention, the ASP or fragment thereof comprises a N-glycan which further comprises a core glycan structure built up by 2 N-acetylglucosamine (GlcNAc) residues in combination with 3 mannose residues and either an alpha-1,3-fucose or alpha-1,6-fucose attached to the Asn-linked GlcNAc residue. Additional glycan branches can be present on the mannose residues (on positions X and/or Y in Structure I), each independently built up of N-acetylglucosamine (GlcNAc), galactose (gal), fucose (fuc), or mannose (man) residues, including combinations thereof. In another embodiment of the invention, the recombinant ASP or fragment thereof comprises an N-glycan comprising the structure according to the confirmation of Structure (I) (FIG. 17).

Preferably, the recombinant ASP or fragment thereof comprises an N-glycan comprising the structure according to the confirmation of Structure (I), wherein X and/or Y are substituted with GlcNac, in particular with Gal-GlcNAc.

In a particular embodiment of the invention, the recombinant ASP is synthetically produced or obtained from an expression system comprising core-modifying fucosyltransferases. Examples of core-modifying fucosyltransferases are fucosyltransferase 8 from mouse or fruit fly in the case of core alpha-1,6-fucose, fucosyltransferase C from Schistosoma mansoni or plant endogenous fucosyltransferase 11 or 12 in the case of core alpha-1,3-fucose.

In another particular embodiment, the recombinant ASP is obtained from an Nicotiana spp. expression system (e.g. a Nicotania benthamiana or Nicotania tabacum expression system), Pichia spp. expression system (e.g. Pichia pastoris expression system), or an insect cell line expression system.

In a further embodiment of the present invention the amino acid sequence of the recombinant ASP or fragment thereof of the invention has at least 90% sequence identity, preferably at least 95% sequence identity, more preferably at least 99%, most preferably 100% sequence identity to the amino acid sequence of ASP of Ostertagia ostertagi, Cooperia oncophora or Teladorsagia circumcincta, in particular wherein the amino acid sequence of the Ostertagia ostertagi, Cooperia oncophora or Teladorsagia circumcincta ASP are as represented by the Genbank accession number CAD23183.1 (in particular amino acids 22-236; AA 1-21 includes a signal sequence MQALIGIAALYLVLVTSNTEA—SEQ ID NO: 13), Genbank accession number CCQ71722.1, and Genbank accession number CBJ15404.1 (in particular amino acids 22-236; AA 1-21 includes a signal sequence MFTPIGIAVLYLALVTPHAKA—SEQ ID NO: 14), respectively.

In another particular embodiment of the present invention the amino acid sequence of the recombinant ASP of Ostertagia ostertagi of the invention is represented by SEQ ID NO: 1, the ASP amino acid sequence of Cooperia oncophora is represented by SEQ ID NOs: 2-4 and the amino acid sequence of the recombinant ASP of Teladorsagia circumcincta is represented by SEQ ID NO: 5.

In a further aspect, the present invention provides a pharmaceutical composition comprising the recombinant ASP or fragment thereof of the invention and a pharmaceutically acceptable carrier and/or excipient.

In a further embodiment, the present invention relates to the pharmaceutical composition comprising the recombinant ASP or fragment thereof of the invention, wherein the pharmaceutical composition is a vaccine.

In another particular embodiment, the invention relates the pharmaceutical composition comprising the recombinant ASP or fragment thereof of the invention additionally comprising an adjuvant.

In yet a further embodiment, the present invention provides a recombinant ASP or fragment thereof of the invention, or the pharmaceutical composition comprising the recombinant ASP or fragment thereof of the invention, for use as a human or veterinary medicine.

In a further particular embodiment, the recent invention provides a recombinant ASP or fragment thereof of the invention, or the pharmaceutical composition comprising the recombinant ASP or fragment thereof of the invention, for use in the treatment, prevention and/or reduction of a parasitic nematode infection in a mammal.

In another particular embodiment, the present invention relates to the recombinant ASP or fragment thereof or pharmaceutical composition comprising the recombinant ASP of the invention for use in the treatment, prevention and/or reduction of a parasitic nematode infection in a mammal, wherein the parasitic nematode belongs to the genus Ostertagia, and/or to the genus Cooperia and/or to the genus Teladorsagia, more in particular wherein the parasitic nematode is Ostertagia ostertagi, Ostertagia leptospicularis, Cooperia oncophora, Cooperia punctate, Cooperia pectinate, Teladorsagia Trifurcata and/or Teladorsagia circumcincta, even more particular wherein the parasitic nematode is Ostertagia ostertagi, Cooperia oncophora or Teladorsagia circumcincta.

In a further embodiment, the present invention relates to a method for producing a recombinant ASP or fragment thereof of the invention, said method comprising the steps of:
a) providing an expression system comprising a fucosyltransferase, more particular a core-modifying fucosyltransferase,
b) introducing in the expression system a fucose sugar, in particular a core α1,3-fucose and/or a core α1,6-fucose,
c) expressing an ASP with the expression system provided in step a) to obtain a recombinant ASP or fragment thereof of the invention.

In yet a further embodiment, the present invention provides a method of treatment, prevention and/or reduction of a parasitic nematode infection in a subject in need thereof, comprising the administration to said subject in need thereof of a recombinant ASP or fragment thereof of the invention or a pharmaceutical composition of the invention.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Amino acid sequences of the Ostertagia ostertagi and Cooperia oncophora ASP. (A) The Oo-ASP-1 amino acid sequence of the recombinant version, and European and North-American isolates is presented. A total of nine positions in the isolate sequences was found to show significant sequence diversity. The corresponding residues are shown in bold. Dotted lines indicate residues with quasi identical polymorphism percentages. (B) Nucleotide sequences encoding and amino acid sequences of C. oncophora double-domain activation-associated secreted proteins.

FIG. 2: Indirect ELISA four peptides vs pooled 3 vaccine groups; Indirect ELISA PUS 028 029 vs individuals 3 vaccine groups.

FIG. 3A: An overview of the synthetic N-glycans present on the array slide.

FIG. 3B: N-glycan recognition by Oo-ASP-1 specific antibodies from native Oo-ASP-1 immunized cows, expressed as mean fluorescence index (MFI) per glycan spot with standard error of the mean based on four technical replicates. There is a clear recognition of all core α1,3-fucosylated N-glycans, whilst α1,6-fucosylation is not always required for antibody recognition.

FIG. 4: Native Oo-ASP, N. benthamiana recombinant ASPs and P. pastoris recombinant ASP were evaluated for their capacity to inhibit the binding of anti-ASP antibodies to the native Oo-ASP. A lower OD405-492 signal corresponds to less antibody binding to the native Oo-ASP coated on the 96-well plate and thus with a higher level of inhibition

FIG. 5: Determination of cell proliferation upon (re-)expose to nASP. Cell proliferation was seen in the CD335+ NK cell group for mostly animals vaccinated with nASP. Proliferation was lower for other cell types and/or vaccine groups.

FIG. 6: Demonstration of systemic IgG1 response in vaccine groups. After the second immunization, a clear (cross)reactive IgG1 response was demonstrated in the nASP and rASP vaccine groups. The increase at necropsy in the QuilA group is a result of the trickle-infection period that all animals went through.

FIG. 7: Determination of local IgG1, IgG2 and IgA response. Similar to the systemic response, a clear (cross)reactive IgG1 and IgG2 response was described in mucus at the time of necropsy. Similarly, this response mostly described for nASP and rASP.

FIG. 8: Fecal egg counts per vaccine group (with SEM) per day. Fecal egg counts per day for each vaccine group, starting 21 days after the first infection.

FIG. 9: Individual egg excretion per day per vaccine group. Per group, all individual animals are allocated to a different symbol to indicate that the animals with higher fecal egg counts are typically the same animals. Importantly, animals that excrete a large number of eggs are not or less present in the nASP and rASP vaccine groups.

FIG. 10: Cumulative fecal egg counts presented in EPG. The AUC per animal was calculated and presented at cumulative EPG. Compared to the QuilA controls, a non-significant reduction of 57% was observed for animals immunized with nASP+QuilA, whereas a similar non-significant reduction of 45% was observed for animals immunized with rASP+QuilA.

FIG. 11: Worm counts of control and vaccine groups. No clear differences in total worms, total adults or total L4 were found if comparing the QuilA controls to the nASP and rASP vaccine groups.

FIG. 12: Worm length determination of control and vaccine groups. No clear differences in worm length were described between the three vaccine groups. If the measured worms were not allocated to the individual animals and instead to the vaccine group to which that animal belongs, a significant reduction in both male and female worm length was observed for nASP vaccinated animals.

FIG. 13: Overview of the synthetic N-glycans. An overview of the synthetic N-glycans present on this array slide, supplemented with its reference name to the data displayed in FIGS. 11 and 12.

FIG. 14: N-glycan recognition by Co-dd-ASP-specific antibodies from native Co-dd-ASP immunized cows. N-glycan recognition by Co-dd-ASP-specific antibodies from native Co-dd-ASP immunized cows, expressed as mean fluorescence index (MFI) per glycan spot with standard error of the mean based on four technical replicates. There is a clear recognition of all core α1,3-fucosylated N-glycans, whilst α1,6-fucosylation is not always required for antibody recognition.

FIG. 15: N-glycan recognition by Co-dd-ASP-specific antibodies from P. pastoris ASP immunized cows. N-glycan recognition by Co-dd-ASP-specific antibodies from P. pastoris ASP immunized cows, expressed as mean fluorescence index (MFI) per glycan spot with standard error of the mean based on four technical replicates. Compared to the native ASP vaccinated animals, N-glycan recognition is more limited.

FIG. 16: Differences between pooled serum samples from the three vaccine groups in peptide recognition. There appear to be no clear differences between pooled serum samples from the three vaccine groups in peptide recognition.

FIG. 17A: Structure (I) FIG. 17B: a) Structure (I)+Gal-GlcNAc (on X or Y)−Core α1,6 fucose. b) Structure (I)+Gal-GlcNAc+GlcNAc (on X or Y (only one is shown))−Core α1,6 fucose. c) Structure (I)+Gal-GlcNAc+Gal-GlcNAc (on X and Y (only one is shown))−Core α1,6 fucose. d) Structure (I)+Gal-GlcNAc (on X or Y (only one is shown))−Core α1,3 fucose. e) Structure (I)+Gal-GlcNAc+GlcNAc (on X or Y (only one is shown))−Core α1,3 fucose. f) Structure (I)+Gal-GlcNAc+Gal-GlcNAc (on X and Y (only one is shown))−Core α1,3 fucose.

FIG. 18: A: N. benthamiana Oo-ASP-1. Core α1,3-fucose, pre-enrichment for terminal galactose. Estimation: Galactosylated N-glycans <5%. B: N. benthamiana Oo-ASP-1. Core α1,3-fucose, post-enrichment for terminal galactose.

FIG. 19: A: N. benthamiana Oo-ASP-1. Core α1,6-fucose, pre-enrichment for terminal galactose. Estimation: Galactosylated N-glycans <5%. B: N. benthamiana Oo-ASP-1. Core α1,6-fucose, post-enrichment for terminal galactose.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. When describing the compounds of the invention, the terms used are to be construed in accordance with the following definitions, unless a context dictates otherwise.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

As used in the specification and the appended claims, the singular forms “a”, “an”, and “the” include plural referents unless the context clearly dictates otherwise.

As already detailed herein above the present invention provides a recombinant activation-associated secreted protein (ASP) or fragment thereof, said ASP or fragment comprising an N-glycan comprising a α1,3-fucose (Fuc) and/or a α1,6-fucose, more specific a core α1,3-fucose and/or a core α1,6-fucose, even more specific a core α1,3-fucose.

Preferably, the invention thus provides a recombinant activation-associated secreted protein (ASP) or fragment thereof, said ASP or fragment comprising an N-glycan comprising a α1,3-fucose, in particular an N-glycan comprising a core α1,3-fucose.

The ability of N-glycans to be recognized by ASP-specific antibodies was investigated with a glycan microarray. All recognized N-glycans of a varied battery of N-glycans contain a core α1,3-fucose, optionally in combination with N-glycans containing a core α1,6-fucose. Core α1,3-fucose, and/or to a lesser extent a core α1,6-fucose, appeared to be essential for a recombinant ASP to be recognized by native ASP induced IgG's.

The ability to artificially induce an immune response with the recombinant ASP comprising N-glycans comprising a core α1,3-fucose or a core α1,6-fucose, in particular a recombinant ASP comprising N-glycans comprising a core α1,3-fucose in combination with a recombinant ASP comprising N-glycans comprising a core α1,6-fucose, thus provides a new use and/or vaccine against gastrointestinal nematodes, such as Ostertagia ostertagi, Ostertagia leptospicularis, Cooperia oncophora, Cooperia punctate, Cooperia pectinate, Teladorsagia Trifurcata and/or Teladorsagia circumcincta.

Acquirement of native ASP requires euthanasia of calves, which impedes large scale production of vaccines. The recombinant ASP of the present invention can be produced by various expression systems enabling large scale production of said vaccines.

The importance of the N-glycosylation in the immune response is further substantiated as the glycan microarray results confirm the ability of antigen-specific antibodies to exclusively bind N-glycans in the absence of the protein. Recognition of N-glycans containing a core α1,3-fucose was surprisingly profound, either on its own or in combination with a core α1,6-fucose.

In particular, the invention therefore further relates to a recombinant activation-associated secreted protein (ASP) or fragment thereof, said ASP or fragment comprising a glycan, in particular an N-glycan, comprising a α1,3-fucose attached to the Asn-linked GlcNAc residue and/or a α1,6-fucose (Fuc) attached to the Asn-linked GlcNAc residue. More specific, the ASP or fragment thereof comprises a core glycan structure built up by 2 N-acetylglucosamine (GlcNAc) residues in combination with 2, in particular 3, mannose residues and either an alpha-1,3-fucose or alpha-1,6-fucose attached to the Asn-linked GlcNAc residue. In a particular embodiment, additional glycans are present on the (outer) mannose residues (on positions X and/or Y in Structure I), each independently built up by or comprising a mannose, galactose, N-acetylgalactosamine, N-acetylglucosamine and/or fucose residue(s), including combinations thereof. More particular, the N-glycan comprises the structure according to Structure (I), including Ia and Ib, as provided herein. In said structure X and/or Y can be absent or when present are independently selected from mannose, galactose, N-acetylgalactosamine, N-acetylglucosamine and fucose, including combinations thereof, in particular X and/or Y are selected from N-acetylglucosamine and galactose. In one embodiment X is absent. In another embodiment Y is absent. In a further embodiment, X comprises GlcNAc and Gal and Y is absent. In another embodiment, Y comprises GlcNAc and Gal and X is absent. In an even further embodiment, X and Y comprise GlcNAc and Gal.

As referred to herein, N-linked glycosylation is the attachment of an oligosaccharide, a carbohydrate consisting of several sugar molecules, sometimes also referred to as glycan, to a nitrogen atom (the amide nitrogen of an asparagine (Asn) residue of a protein), in a process called N-glycosylation.

The recombinant ASP or fragment of the invention may also be referred to as isolated ASP or fragment.

In the context of the present invention the term “recombinant” refers to being synthetically produced or expressed in an expression system which is not the native expression system. A recombinant protein or fragment thereof should thus be interpreted as a protein expressed in an expression system which is different from the expression system it is natively expressed in. The term “recombinant” also includes molecules formed by laboratory methods of genetic recombination, which bring together genetic material from different sources, thereby creating sequences which would not otherwise be found in the native genome.

Also, in the context of the present invention, the term “recombinant ASP of the invention” should be interpreted as a recombinant activation-associated secreted protein or fragment thereof comprising at least one glycan, in particular a N-glycan, comprising a core α1,3-fucose and/or a core α1,6-fucose (Fuc), in particular a core α1,3-fucose. In one embodiment, the recombinant ASP or fragment comprises two, three, four or more N-glycans having a core α1,3-fucose and/or core α1,6-fucose (Fuc), in particular a core α1,3-fucose.

The term “isolated” is used to indicate that a cell, peptide or nucleic acid is separated from its native environment. Isolated proteins, peptides and nucleic acids may be substantially pure, i.e. essentially free of other substances with which they may bound in nature.

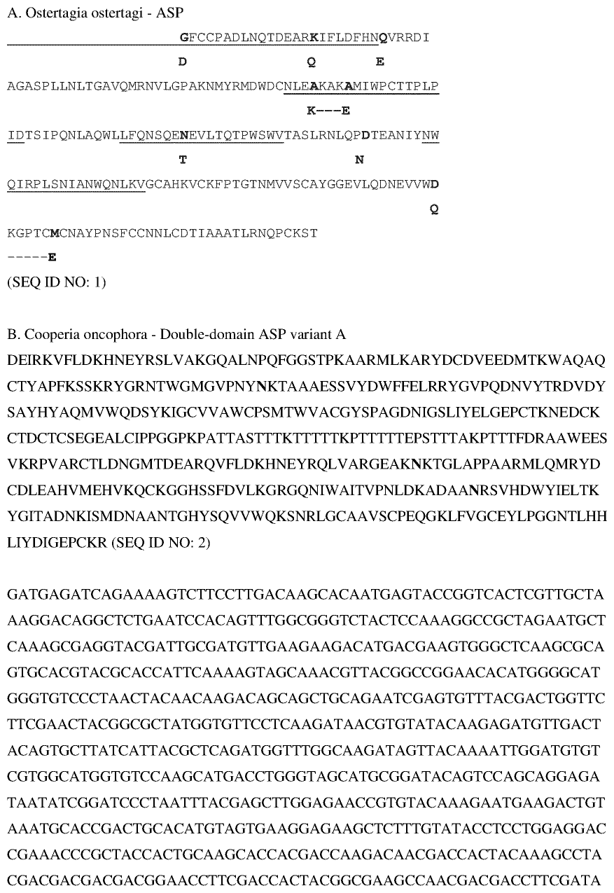

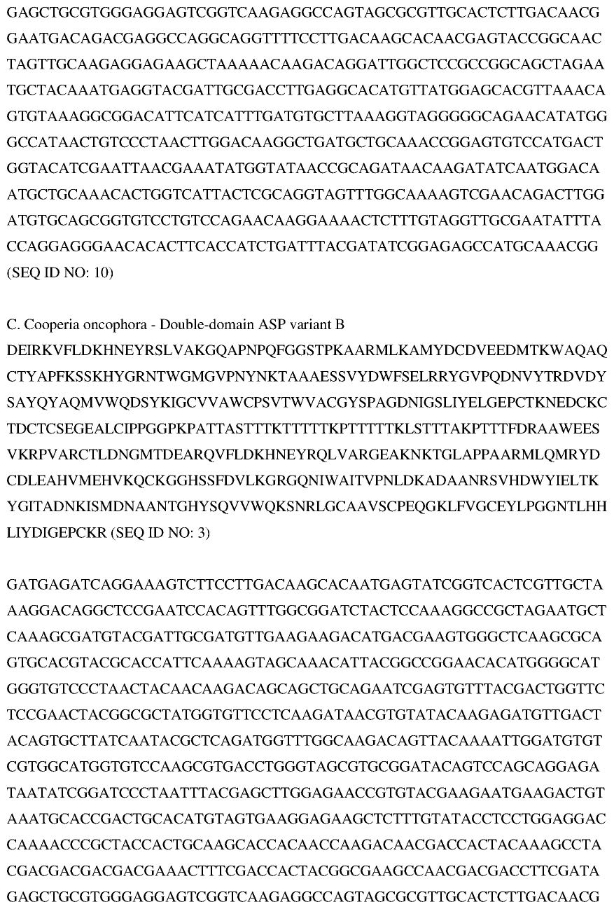

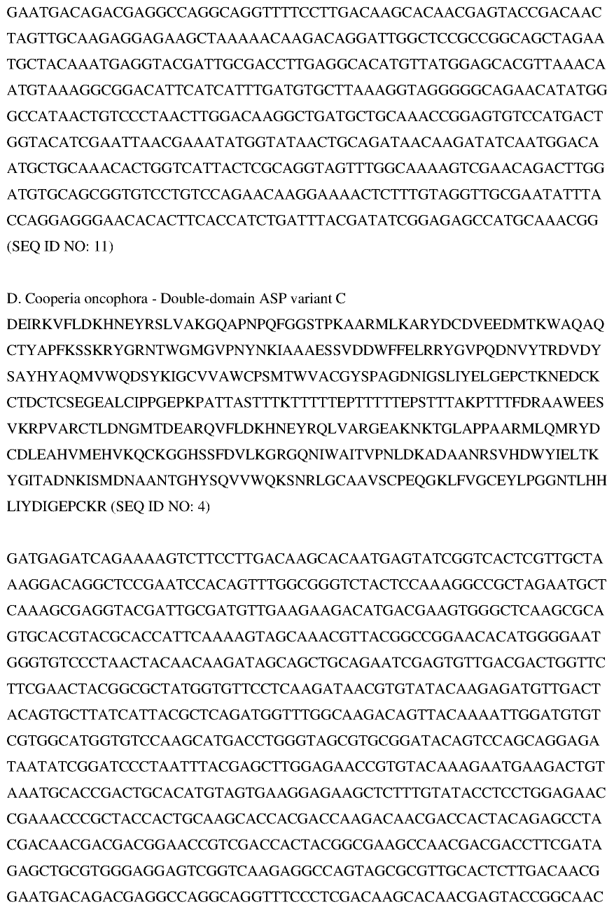

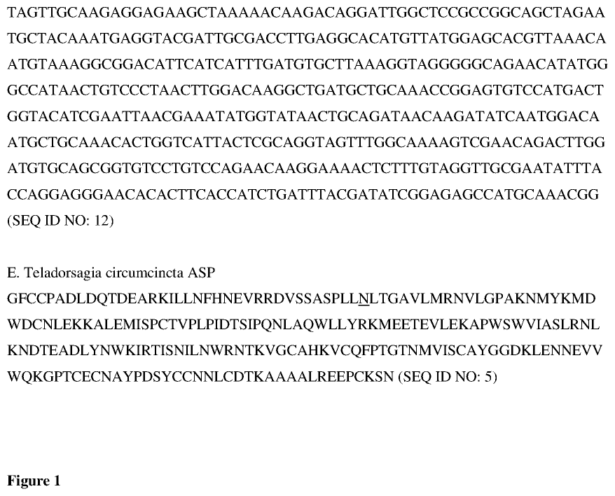

In one embodiment of the present invention, the amino acid sequence of the recombinant ASP or fragment thereof of the invention has at least 90% sequence identity (thus including at least 91%, 92%, 93%, 94%, 95%, 96%, 97%, 98% and 99% sequence identity), preferably at least 95% sequence identity, more preferably at least 99%, most preferably 100% sequence identity with the amino acid sequence of ASP of Ostertagia ostertagi, Cooperia oncophora, or Teladorsagia circumcincta, wherein the amino acid sequence of the Ostertagia ostertagi, Cooperia oncophora, and Teladorsagia circumcincta ASP are in particular as provided in FIG. 1, or as present in Genbank with accession number CAD23183.1 (in particular excluding the signal sequence), accession number CCQ71722.1, or accession number CBJ15404.1 (in particular excluding the signal sequence), respectively. In another embodiment, the nucleic acid sequence encoding the recombinant ASP or fragment thereof as provided herein has at least 90% sequence identity (thus including at least 91%, 92%, 93%, 94%, 95%, 96%, 97%, 98% and 99% sequence identity), preferably at least 95% sequence identity, more preferably at least 99%, most preferably 100% sequence identity with the nucleic acid sequence of ASP of Ostertagia ostertagi, Cooperia oncophora or Teladorsagia circumcincta, as represented by accession number CAD23183.1 (in particular excluding the signal sequence), accession number CCQ71722.1, or accession number CBJ15404.1 (in particular excluding the signal sequence), respectively.

In a further aspect of the invention, the amino acid sequence of the recombinant ASP of the invention of Ostertagia ostertagi is represented by SEQ ID NO: 1, the amino acid sequence of the recombinant ASP of the invention of Cooperia oncophora is represented by SEQ ID NOs: 2-4, and the amino acid sequence of the recombinant ASP of the invention of Teladorsagia circumcincta is represented by SEQ ID NO: 5. Optional N-terminus and/or C-terminus sequences for C. oncophora are characterized by the amino acids LCSLDNGMT (SEQ ID NO: 6) and DEDCKCSSCRCSTQLSMCINPN (SEQ ID NO: 7), respectively.

The percentage identity of nucleic acid and polypeptide sequences can be calculated using commercially available algorithms, which compare a reference sequence with a query sequence. The following programs (provided by the National Center for Biotechnology Information) may be used to determine homologies/identities: BLAST, gapped BLAST, BLASTN and PSI BLAST, which may be used with default parameters.

The term “fragment” as used herein refers to partial amino acid sequences (and nucleic acid sequences coding therefore) having at least one immunologic or immunogenic property in common with the native molecule, and hence e.g. the ability to generate antibodies in an immunized animal.

Thus, the full-length sequence of the ASP is not necessary, and neither is the 100% identity to SEQ ID Nos provided herein, meaning that one or more amino acid modifications are possible. For example, the fragment may have up to 30 amino acids removed from the N- or C-terminal ends of the protein, e.g. up to about 1, 5, 10, 15, 20, or 30 amino acids removed. As noted elsewhere in the present disclosure, 90% sequence identity is likely to be sufficient to provide suitable level of antibody production as long as the protein comprises at least one N-glycan comprising a α1,3-fucose and/or a α1,6-fucose (Fuc) as provided herein. As used herein, the term “amino acid modification” refers to an amino acid addition, amino acid deletion, and/or to an amino acid substitution as compared to the reference sequence. Preferably, said one or more amino acid substitution is a ‘conservative’ amino acid substitution, i.e. the substitution of an amino acid by another amino acid of the same class, in which the classes are as follows: Class Amino acid examples

| Class | Amino acid examples |
|---|---|
| Nonpolar | Ala, Val, Leu, Pro, Met, Phe, Trp, Ile |
| Uncharged polar | Gly, Ser, Thr, Cys, Tyr, Asn, Gln |
| Acidic | Asp, Gly |
| Basic | Lys, Arg, His |

The differing amino acids can be conservative substitutions, and/or are located outside of immunodominant epitope(s) of the ASP fragment. As used herein the term “immunogenic fragment” in regard to the ASP protein is a fragment of that protein that is immunogenic, i.e., capable of specifically interacting with an antigen recognition molecule of the immune system, such as an immunoglobulin (antibody) or T cell antigen receptor. Preferably, an immunogenic fragment of the present invention is immunodominant for antibody and/or T cell receptor recognition. In a particular embodiment, an immunogenic fragment as referred to herein is a fragment of the ASP provided herein that retains at least 50%, 60%, 70%, 80%, or 90% of the immunogenicity of the full-length protein. Fragments can be as small as 8 amino acids or at the other extreme, be large fragments that are missing as little as a single amino acid from the full-length protein as provided herein. In a particular embodiment the fragment comprises 8 to 249 amino acid residues of the full-length protein. In other embodiments, the fragment comprises or consist of 10 to 200, 10 to 150, 10 to 120, to 100, or 10 to 50 amino acid residues. Such fragments will include at least one epitope (or antigenic determinant) of the native molecule. In one embodiment, they have a length of at least 8 amino acids, preferably at least 10, 11, 12, 13, 14, 15 or 20 amino acids.

A suitable non-limiting example of such fragments for Ostertagia ostertagi ASP is represented by a sequence comprising at least Asn9 and/or Asn37 (including the N-glycans of the invention), said fragment e.g. comprising or consisting of the amino acid sequence as represented by SEQ ID NO: 8 (GFCCPADLNQTDEARKIFLDFHNQVRRDIAGASPLLNLTGAV).

In a specific embodiment, the recombinant ASP or fragment for O. osteragi as provided herein comprises or consists of the amino acid sequence as represented by SEQ ID NO: 9 (GFCCPADLNQTDEARKIFLDFHN), and hence comprising at least Asn9 (including the N-glycans of the invention).

A suitable non-limiting example of a suitable fragment for Cooperia oncophora dd-ASP is represented by a sequence comprising at least either one of Asn83, Asn277 or Asn340, according to the numbering in SEQ ID NO: 2-4. In a specific embodiment, the recombinant ASP or fragment for C. oncophora as provided herein comprises at least the amino acids Asn83 and Asn277, or Asn277 and Asn340, or Asn83, Asn277 and Asn340.

A suitable non-limiting example of a suitable fragment for Teladorsagia circumcincta ASP (Tc-ASP) is represented by a sequence comprising at least Asn37 (including the N-glycans of the invention), according to the numbering in SEQ ID NO: 5.

Fragments comprising two or more of the specified asparagine residues (Asn), selected from one species or a combination of different species, can be combined as a mixture or in a fusion protein.

The term “fusion protein” refers to a polypeptide sequence translated from a nucleic acid transcript generated by combining a first nucleic acid sequence that encodes a first peptide fragment as provided herein and at least a second nucleic acid that encodes a second peptide as provided herein, where the fusion protein is not a naturally occurring protein. The nucleic acid construct may encode two, three, four or more peptides that are joined in the fusion protein, optionally separated by a linker sequence. Methods for generating fusion proteins are generally known to a skilled person.

A “glycan” as used herein generally refers to glycosidically linked monosaccharides, oligosaccharides and polysaccharides. Hence, carbohydrate portions of a glycoconjugate, such as a glycoprotein, glycolipid, or a proteoglycan are referred to herein as a “glycan”. Glycans can be homo- or heteropolymers of monosaccharide residues, and can be linear or branched. The term “N-glycan” or alternatively ‘N-linked glycan’ relates to a glycan attached to the nitrogen atom of an asparagine (Asn) side chain. Such N-glycans are attached to eukaryotic proteins and play a major role in the structure and function of said proteins. Typical N-glycans may be selected from the list comprising N-acetylgalactosamine, galactose, neuraminic acid, N-acetylglucosamine, fucose, mannose, or other monosaccharides.

Another aspect of the present invention is that the N-glycan of the recombinant ASP or fragment thereof, at least comprises N-acetylglucosamine (GlcNAc), mannose (Man) and fucose (Fuc). In a broad sense, the invention provides an ASP or fragment thereof, comprising a carbohydrate compound, in particular an N-glycan, according to the formula: N-acetylglucosamine(X) and mannose, which is in shortened notation: GlcNAc(X)Man, and wherein X is α1,3-fucose or a α1,6-fucose.

More specific, the carbohydrate compound comprises at least two GlcNAc residues and at least two, in particular at least three Man residues. In a further embodiment, the carbohydrate compound further comprises a galactose.

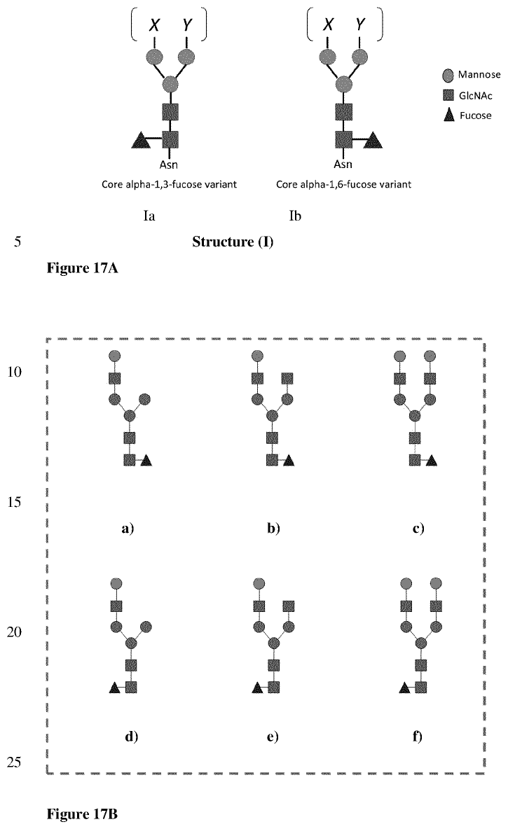

In a specific embodiment, the ASP or fragment carries two hybrid N-glycan structures with a complex-1,3-arm and an unprocessed-1,6-arm. The 1,3-arm constitutes of an N-acetylglucosamine residue and a terminal galactose residue. Furthermore, the innermost N-acetylglucosamine residue of the N-linked glycans carry either alpha 1,6- or alpha 1,3-fucose residue. The N-glycan according to the invention further preferably comprises fucose, GlcNAc, Man and Gal in the conformation of structure (I) (FIG. 17A).

Examples of recombinant ASPs of the invention are provided in FIG. 17B.

In a further specific embodiment, the recombinant ASPs of the invention comprise or consist of N-glycan structures according to FIG. 17B, wherein the N-glycan structure is structure (I)+Gal-GlcNAc (on X or Y) and core α1,6 fucose or structure (I)+Gal-GlcNAc+GlcNAc (on X or Y) and core α1,6 fucose or structure (I)+Gal-GlcNAc+Gal-GlcNAc (on X and Y) and core α1,6 fucose or structure (I)+Gal-GlcNAc (on X or Y) and core α1,3 fucose or structure (I)+Gal-GlcNAc+GlcNAc (on X or Y) and core α1,3 fucose or structure (I)+Gal-GlcNAc+Gal-GlcNAc (on X and Y) and core α1,3 fucose.

For the recombinant Oo-ASP-1 (SEQ ID NO: 1), glycans are present on Asn9 and Asn 37, which are identical to those found on endogenous Oo-ASP.

For the recombinant Co-dd-ASP (SEQ ID NO: 2-4), glycans are present on Asn83, Asn277 and Asn340.

For the recombinant Tc-ASP (SEQ ID NO: 5), glycans are present on Asn39.

The chemical structure of the glycans associated with the ASP antigen according to the invention, is in accordance with the common nomenclature in carbohydrate biochemistry.

Fucosylation is a type of glycosylation and can be defined by the process of adding fucose sugar units to a molecule. Expression systems having the ability to fucosylate are therefore able to provide the recombinant ASP or fragments of the present invention. Enzymes able to perform such fucosylation processes are fucosyltransferases. These enzymes can be endogenously present in the expression host or can be co-expressed with the ASP protein. In an embodiment of the invention, the recombinant ASP or fragment thereof of the invention is synthetically produced or obtained from an expression system comprising a specific fucosyltransferase that transfers either alpha 1,6- or alpha 1,3-fucose, in particular a core α1,3-fucose, to the core of the N-glycan. In a specific embodiment said fucosyltransferase is a core-modifying fucosyltransferase. These can be fucosyltransferase 8 from mouse or fruit fly in the case of core alpha-1,6-fucose, and fucosyltransferase C from Schistosoma mansoni or plant endogenous fucosyltransferase 11 or 12 in the case of core alpha-1,3-fucose.

According to a further embodiment of the present invention the ASP protein is produced by the expression of a polynucleotide as described herein. Suitable vectors for expression of proteins are plasmids, bacteriophages, cosmids, viruses, minichromosomes or stably integrating vectors.

Generally, these vectors have the property of autonomous replication except for the stably integrating vectors which insert themselves in the genetic material of the host cell and replicate with host's genetic material. Suitable host cells for the expression of proteins may either be prokaryotic or eukaryotic, such as but not limited to bacteria such as Escherichia coli, yeasts such as Saccharomyces cerevisiae and Pichia pastoris, mycoplasma's, algae, plant cells such as Arabidopsis thaliana and Nicotiana spp., such as Nicotania benthamiana or Nicotania tabacum, vertebrate cells, or baculovirus/insect cells; the plant or animals cells may be cultivated in vitro or may form part of an intact plant or animal, respectively.

According to a particular embodiment, the (host) cell of the present invention is a glyco-engineered cell. A “glyco-engineered cell” refers to a cell that has been genetically modified so that it expresses proteins with an altered N-glycan structure as compared to a non-engineered cell or expression system. Typically, this includes the use of specific enzymes involved in the glycosylation pathway. In general, sugar chains in N-linked glycosylation may be divided in three types: high-mannose (typically yeast), complex (typically mammalian) and hybrid type glycosylation. The different types of N-glycosylation are all well known to the skilled person and defined in the literature. Considerable effort has been directed towards the identification and optimization of strategies for the engineering of eukaryotic cells that produce glycoproteins having a desired N- and/or O-glycosylation pattern and are known in the art (van der Kaaij et al., 2022; Ma et al. 2020—incorporated herein by reference). Enzymes needed for complex glycosylation include, but are not limited to: N-acetylglucosaminyl transferase I, N-acetylglucosaminyl transferase II, mannosidase II, galactosyltransferase, fucosyltransferase and sialyltransferase, and enzymes that are involved in donor sugar nucleotide synthesis or transport. Still other glyco-engineered cells, in particular yeast cells, that are envisaged here are characterized in that at least one enzyme involved in the production of high mannose structures (high mannose-type glycans) is not expressed. Enzymes involved in the production of high mannose structures typically are mannosyltransferases. In particular, alpha-1,6-mannosyltransferases Ochlp, Alg3p, alpha-1,3-mannosyltransferase of the Mnnlp family, beta-1,2-mannosyltransferases may not be expressed. Thus, a cell can additionally or alternatively be engineered to express one or more enzymes or enzyme activities, which enable the production of the particular N-glycan structures of the invention at a high yield. Such an enzyme can be targeted to a host subcellular organelle in which the enzyme will have optimal activity, for example, by means of signal peptide not normally associated with the enzyme. It should be clear that the enzymes described herein and their activities are well-known in the art.

The recombinant polynucleotide may contain as an insert a complete polynucleotide coding for the ASP or a fragment thereof. Bacterial, yeast, fungal, insect, plant and vertebrate cell expression systems (e.g. CHO cells) are very frequently used systems. Such systems are well known in the art and generally available.

In particular, the invention relates to the recombinant ASP of the invention which is obtained from the expression systems comprising a fucosyltransferase such as a Nicotiana spp. expression system (e.g. Nicotania benthamiana or Nicotania tabacum), Pichia pastoris expression system, or an insect cell line expression system.

In a further aspect, the invention provides a method to produce a recombinant ASP or fragment thereof, said method comprising the steps of introducing an expression vector comprising a nucleotide sequence provided herein in a suitable expression host, expressing and isolating said protein or fragment of the invention. By the term “a suitable cell” a higher eukaryotic cell, such as a mammalian cell or a plant cell, a lower eukaryotic cell, such as a filamentous fungus cell or a yeast cell, said cell being optionally glyco- engineered, is envisaged as explained above.

Particularly envisaged herein is the production of a recombinant ASP or fragment provided herein, wherein said protein or fragment is glycosylated and comprises one or more glycans, in particular N-glycans.

For example, an ASP or fragment, wherein said protein or fragment is N-glycosylated and comprises a mixture of lycans with a GlcNAc, Gal and α1,3-fucose and/or α1,6-fucose as provided herein can typically be obtained by expression in a higher or lower eukaryotic glyco-engineered cell known to the skilled person.

In another embodiment, the invention relates to a method for producing a recombinant ASP or fragment of the invention, said method comprising the steps of:
a) providing an expression system comprising a fucosyltransferase, more particular a core modifying fucosyltransferase,
b) introducing in the expression system a fucose sugar, in particular a core α1,3-fucose and/or a core α1,6-fucose, more in particular a core α1,3-fucose,
c) expressing an ASP with the expression system provided in step a) to obtain a recombinant ASP of the invention.

In this method, the expression system comprising a fucosyltransferase can be Nicotiana benthamiana expression system, Pichia pastoris expression system, or an insect cell line expression system. A fucosyltransferase is an enzyme that transfers an L-fucose sugar from a GDP-fucose (guanosine diphosphate-fucose) donor substrate to an acceptor substrate. The acceptor substrate can be another sugar such as the transfer of a fucose to a core GlcNAc (N-acetylglucosamine) sugar as in the case of N-linked glycosylation, or to a protein, as in the case of O-linked glycosylation produced by O-fucosyltransferase. In a particular embodiment, the invention comprises a recombinant ASP or fragment obtained by said method. Furthermore, the recombinant ASP of the invention as obtained from this method can be comprised in a pharmaceutical composition. The recombinant ASP of the invention and/or the pharmaceutical composition comprising the recombinant ASP of the invention can be used as a medicine, preferably as a vaccine to treat, prevent or reduce infection of parasitic nematodes provided herein, in particular Ostertagia ostertagi, Cooperia oncophora and/or Teladorsagia circumcincta.

In a further embodiment, the invention relates to a pharmaceutical composition comprising a recombinant ASP or fragment thereof according to invention, and a pharmaceutically acceptable carrier and/or excipient. In a particular embodiment, the pharmaceutical composition comprises a recombinant ASP or fragment thereof, said ASP or fragment comprising an N-glycan comprising a (core) α1,3-fucose or a (core) α1,6-fucose, and a pharmaceutically acceptable carrier and/or excipient. In a further embodiment, the pharmaceutical composition comprises a recombinant ASP or fragment thereof, said ASP or fragment comprising an N-glycan comprising a (core) α1,3-fucose, a recombinant ASP or fragment thereof, said ASP or fragment comprising an N-glycan comprising a (core) α1,6-fucose, and a pharmaceutically acceptable carrier and/or excipient. In a further specific embodiment, the composition comprises one or more ASPs or fragments of the invention (Structure I), further including additional glycans present on the (outer) mannose residues (on positions X and/or Y in Structure I), each independently built up by or comprising a mannose, galactose, N-acetylgalactosamine, N-acetylglucosamine and/or fucose residue(s). More particular, the N-glycan comprises the structure according to Structure (I), including Ia and Ib, as provided herein. In said structure X and/or Y can be absent or when present are independently selected from mannose, galactose, N-acetylglucosamine and fucose, including combinations thereof, in particular X and/or Y are selected from N-acetylglucosamine and galactose. In one embodiment X is absent. In another embodiment Y is absent. In a further embodiment, X comprises GlcNAc and Gal and Y is absent or in the alternative Y comprises GlcNAc and Gal and X is absent. In an even further embodiment, X and Y comprise GlcNAc and Gal.

In a specific embodiment, the composition comprises a combination of one or more, in particular 2, 3, 4, 5, 6, 7, 8, 9 or all of the ASPs as presented in FIG. 17B.

The term “pharmaceutically acceptable carrier” or “pharmaceutically acceptable excipient” means solvents, dispersion media, emulsifying agents, disintegrants, isotonic agents, absorption delaying agents, and the like, that are compatible with pharmaceutical administration. Effective amounts of preservatives can also be included into the formulations. Suitable preservatives include, without limitations, benzalkonium chloride (0.003-0.03% w/v); chlorobutanol (0.3-0.9% w/v); parabens (0.01-0.25% w/v) and thimerosal (0.004-0.02% w/v). The use of such media and agents for pharmaceutically active substances, and the determination of an effective amount, is well known in the art.

For these purposes, the pharmaceutical composition of the present invention may be formulated by means known in the art into the form of, for example, tablets, pellets, capsules, aqueous or oily solutions, suspensions, emulsions, creams, ointments, gels, nasal sprays, suppositories, finely divided powders or aerosols or nebulizers for inhalation, and for parenteral use (including intravenous, intramuscular or infusion) sterile aqueous or oily solutions or suspensions or sterile emulsions.

In practicing the present methods, a vaccine or composition of the present invention is administered preferably via intramuscular or subcutaneous routes, although other routes of administration can be used as well, such as e.g. by oral, intranasal (e.g. aerosol or other needleless administration), intra-lymph node, intradermal, intraperitoneal, rectal or vaginal administration, or by a combination of routes. The formulation of the composition or the vaccine can be made in various forms depending upon the route of administration. For example, the compositions can be made in the form of sterile aqueous solutions or dispersions suitable for injectable use, or made in lyophilized forms using freeze-drying techniques. Lyophilized immunogenic compositions are typically maintained at about 4° C., and can be reconstituted in a stabilizing solution, e.g. saline or and HEPES, with or without adjuvant.

Boosting regimens may be required and the dosage regimen can be adjusted to provide optimal immunization. Immunization protocols can be optimized using procedures well known in the art. A single dose can be administered to animals, or, alternatively, two, three or more inoculations can take place with intervals of two to ten weeks. Depending on the age of the animal, the immunogenic or vaccine composition can be re-administered. For example, the present invention contemplates the vaccination of healthy calves (3-12 months of age) 6 and/or 3 weeks prior to their first grazing season and revaccination at the beginning of the first grazing season.

The ASP or fragment thereof of the present invention for use in the treatment, prevention and/or reduction of a nematode infection, in particular with Ostertagia ostertagi, Cooperia oncophora and/or Teladorsagia circumcincta, is preferably dosed in a therapeutically effective amount. The term “therapeutically effective amount” refers to an amount sufficient to elicit an immune response and/or to confer protection in the animal to which it is administered. The immune response may comprise, without limitation, induction of cellular and/or humoral immunity. The amount of a vaccine that is therapeutically effective may vary depending on the condition of the animal (e.g. ruminant or cattle) and/or the degree of infection, and can be determined by a veterinary physician. “Protection” (and related terms such as “immunoprotection” and “immunoprotective”) as used herein, means to induce an immune response for aiding in preventing, ameliorating, reducing sensitivity for, or treatment of a disease or disorder resulting from infection with a parasitic nematode. The term “reduction” relates to reducing susceptibility for nematode infection. In the context of the present invention and as demonstrated herein, this means causing the target subject to show a reduction in the number and/or size of adult and/or juvenile parasites, the number of eggs or the intensity of clinical signs caused by the parasitic nematode infection. This may be the result of a reduced colonization or of a reduced infection rate by the parasite, leading to a reduction in the number or the severity of lesions, shedding and effects that are caused by the parasite or by the subject's response thereto.

The ability of the recombinant ASP of the invention to induce an immune response to parasitic nematodes such as Ostertagia ostertagi, Cooperia oncophora, and/or Teladorsagia circumcincta, allows that the pharmaceutical composition of the invention comprising this recombinant ASP can be used as a vaccine against these nematodes, in particular against nematodes of the genera Ostertagia, Cooperia and Teladorsagia, more in particular against Ostertagia ostertagi Cooperia oncophora and/or Teladorsagia circumcincta. In a preferred embodiment, the pharmaceutical composition of the invention is thus a vaccine.

To provide such immunity against said nematodes, in particular Ostertagia ostertagi, Cooperia oncophora and/or Teladorsagia circumcincta, the vaccine may optionally further comprise an adjuvant. Therefore, in a further aspect of the invention, the pharmaceutical composition of the invention comprises an adjuvant.

Adjuvants are known to act in a number of different ways to enhance the immune response. In general, immunomodulatory adjuvants cause a general up-regulation of certain cytokines and a concomitant down regulation of others leading to a cellular Th1 and/or a humoral Th2 response.

Suitable adjuvants include, without limitations, oil emulsions (such as water-in-oil, oil-in-water, watier-in-oi-in-water), Complete Freund's adjuvants, saponins such as for example Quil A, ISCOMs (complexes of saponins, sterols and phospholipids), Aluminum compounds including Aluminum phosphate and Aluminum hydroxide, mycobacterial cell wall extracts, MPL-A, quaternary ammonym compounds such as dimethyl dioctadecyl ammonium bromide (DDA), acrylic acid based polymers such as carbomers including CARBOPOl®, glycolipids such as BAY® R1005 and oligonucleotides containing CpG motifs. Combinations of these compounds are also envisioned. Preferably, the composition as described herein is an immunogenic composition. By “immunogenic” is meant the capacity to provoke an immune response in a subject against the pathogen/parasite. The present invention accordingly provides compositions for use in eliciting an immune response which may be utilized as a vaccine, in particular against nematodes belonging to the genus Ostertagia, and/or to the genus Cooperia and/or to the genus Teladorsagia, more in particular against the nematodes Ostertagia ostertagi, Ostertagia leptospicularis, Cooperia oncophora, Cooperia punctate, Cooperia pectinate, Teladorsagia Trifurcata and/or Teladorsagia circumcincta, even more particular against the nematodes Ostertagia ostertagi Cooperia oncophora and/or Teladorsagia circumcincta. The immune response can be a cellular immune response mediated primarily by NK cells, cytotoxic T-cells, and/or a humoral immune response mediated primarily by helper T-cells, which in turn activates B-cells leading to antibody production. More specific, by “eliciting or inducing an immune response” is meant that an antigen stimulates synthesis of specific IgG1 antibodies and/or cellular proliferation as measured by, for example, 3H thymidine incorporation of NK cells, T-cells and B-cells.

In one embodiment, administering the composition or vaccine of the invention elicits an immune response that results in a reduction in mean cumulative fecal egg count of at least about 30%, 40%, 45% or 50% in an animal in relation to a non-vaccinated (e.g. adjuvant alone) control animal. Preferably, the level of the decrease is about 55%, more preferably about 60% and most preferably, about 70% or greater. In a specific embodiment, the reduction in mean cumulative fecal egg count is present for at least 4 weeks after the first administration of the recombinant ASP or fragment, in particular for at least 5 weeks, 6 weeks, 7 weeks or 8 weeks. Hence the immune response confers some beneficial, protective effect to the subject against a subsequent challenge with the infectious agent. More preferably, the immune response prevents the onset of or ameliorates at least one symptom of a disease associated with the infectious agent, or reduces the severity of at least one symptom of a disease associated with the infectious agent upon subsequent challenge.

Given the beneficial medical properties of the composition of the invention, the present invention relates, according to another aspect, to the recombinant ASP or the pharmaceutical composition of the invention, for use as a human or veterinary medicine.

The immune response induced by the recombinant ASP or fragment, or pharmaceutical composition of the invention can treat, prevent or reduce infection of parasitic nematodes, such as nematodes belonging to the genus Ostertagia, and/or to the genus Cooperia and/or to the genus Teladorsagia, more in particular the parasitic nematodes Ostertagia ostertagi, Ostertagia leptospicularis, Cooperia oncophora, Cooperia punctate, Cooperia pectinate, Teladorsagia Trifurcata and/or Teladorsagia circumcincta, even more particular the parasitic nematodes Ostertagia ostertagi, Cooperia oncophora and/or Teladorsagia circumcincta. A further aspect of the invention is therefore that the recombinant ASP or fragment or the pharmaceutical composition of the invention can be used in the treatment, prevention and/or reduction of a parasitic nematode infection in a subject, in particular a mammal.

By “subject” or “host” is meant any mammal/animal that has or is susceptible to a nematode infection, in particular susceptible to infection with nematodes belonging to the genus Ostertagia, and/or to the genus Cooperia and/or to the genus Teladorsagia, more in particular with Ostertagia ostertagi, Ostertagia leptospicularis, Cooperia oncophora, Cooperia punctate, Cooperia pectinate, Teladorsagia Trifurcata and/or Teladorsagia circumcincta, even more in particular with Ostertagia ostertagi, Cooperia oncophora, and/or Teladorsagia circumcincta, such as humans or non-human animals, in particular ruminants, more in particular cattle The term “ruminants” includes many domesticated animals, or animals that otherwise are of agricultural, veterinary or economic importance (e.g. domestic herds), such as sheep, goats, cattle, bison, yaks, water buffalo, deer, camels, llamas, alpacas, as well as various wild animals. “Small ruminants” are understood to include sheep, goats, and deer. The term “cattle” refers to bovine animals including but not limited to steer, bulls, cows, and calves.

In a preferred embodiment, the recombinant ASP or fragment or the pharmaceutical composition of the invention can be used in the treatment, prevention and/or reduction of nematode infections, in particular infections with nematodes belonging to the genus Ostertagia, and/or to the genus Cooperia and/or to the genus Teladorsagia, more in particular with Ostertagia ostertagi, Ostertagia leptospicularis, Cooperia oncophora, Cooperia punctate, Cooperia pectinate, Teladorsagia Trifurcata and/or Teladorsagia circumcincta, even more in particular with Ostertagia ostertagi, Cooperia oncophora, or Teladorsagia circumcincta.

In a further embodiment, the invention relates to a method of treatment, prevention and/or reduction of a parasitic nematode infection in a subject in need thereof, comprising the administration to said subject in need thereof of a recombinant ASP or fragment or of a pharmaceutical composition of the invention.

The invention will now be illustrated by means of the following examples, which do not limit the scope of the invention in any way.

### EXAMPLES

### Example 1

In a first line of investigation on what could be the reason(s) for the inability of the Pichia-produced Oo-ASP-1 in inducing a proper immune response and protection after vaccination of calves (Gonzalez-hernandez, 2016) we looked into the amino acid sequences (i.e. primary structure) of both the endogenous and recombinant molecules. The structural features of the Pichia-produced recombinant Oo-ASP-1 and two forms of its endogenous counterpart, one purified from O. ostertagi excretory/secretory material, were explored via gel filtration (GF-Oo-ASP-1) and the other through a thiol-Sepharose protocol (TS-Oo-ASP-1).

In terms of primary structure in endogenous Oo-ASP-1 we have identified nine hotspots with significant amino acid substitution frequencies, however these were found not to be part of any predicted B-cell epitopes. Antibody binding experiments confirmed that these substitutions play no significant role in antibody recognition. This was confirmed after production of a recombinant Oo-ASP-1 version (dubbed Oo-ASP-1-sub) carrying all nine abovementioned substitutions (that is, G1D, K16Q, Q24E, A65K, A69E, N100T, D121N, D179Q and M185E) and including it in an inhibition ELISA setup.

Following sequence analysis and its impact on antibody recognition, we sought to verify whether recombinant production of Oo-ASP-1 may have introduced any aberrant secondary structural features. Therefore, circular dichroism (CD) experiments were conducted on endogenous TS-Oo-ASP-1 and recombinant Oo-ASP-1. In addition, an LC-MS approach was applied to both ASP versions.

Whereas α-helical and β-sheet contents and dimerization propensity were demonstrated to be identical between endogenous and recombinant Oo-ASP-1, we found the recombinant ASP bearing all CAP protein hallmark disulphide bonds, which was not the case for the endogenous ASP. As we attributed this to its thiol-Sepharose-based purification protocol, a gel filtration-purified endogenous ASP was included in the study, displaying structural properties identical to the recombinant version. As the disulphide bonding pattern of recombinant Oo-ASP is near identical to its endogenous counterpart, the underlying cause for the lack of protection should be sought in other tertiary and/or quaternary structural traits, such as N-glycosylation.

### Example 2

### Obtaining Endogenous and Recombinant ASP

As described in a previous study by Geldhof et al. (2000), ES-material was obtained from adult O. ostertagi worms collected from the abomasum of infected calves. To purify Oo-ASP, the ES-fraction was applied onto a Superdex 200 16/70 column (GE Healthcare Bio-Sciences AB; Uppsala, Sweden) at a flow rate of 1 ml/min to perform gel filtration chromatography (Borloo et al., 2013a). Recombinant versions of Oo-ASP were expressed in P. pastoris and purified as described in Borloo et al. (2013b).

### Peptide Microarray

A peptide microarray was carried out by Pepscan Presto BV. in order to obtain insights in protein recognition by protective IgG antibodies. An array of linear peptide epitopes was generated by splitting the amino acid sequence of Oo-ASP in overlapping fragments in silico on a solid support called “mini-card”. Additionally, conformational epitopes were constructed through the Chemically Linked Peptides on Scaffolds (CLIPS) technology (Timmerman et al., 2007), also in overlapping fragments, which allows mimicking of secondary structure elements, such as loops, α-helixes and β-strands. Constructs that form an incomplete part of the antibody epitope may be recognised by antibodies, albeit with a lower affinity, whereas constructs that are not a part of this epitope are not recognised. The overlapping battery of ASP peptides was synthesized using solid-phase 9-fluorenylmethoxycarbonyl (Fmoc) synthesis and an amino functionalised polypropylene support was obtained by grafting with a proprietary hydrophilic polymer formulation. This was followed by a reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups through the addition of trifluoroacetic acid (TFA). A standard Fmoc-peptide synthesis was used to synthesize peptides on the amino-functionalised solid support.

The binding of serum antibodies to each peptide was evaluated in a pepscan-based ELISA. This was conducted with serum samples from cattle vaccinated with either native Oo-ASP (n=5), P. pastoris Oo-ASP (n=5) or only QuilA adjuvants as control (n=5), which were obtained one week after the third immunisation. In order to prevent deviations in antibody binding, all pre-coat conditions (antibody concentration and the relative amount of competing proteins in the ELISA buffer) were equalised between the different serum samples. At first, array cards we the peptide arrays were incubated overnight at 4° C. with the serum antibodies in PBS containing 5% (v/v) horse serum, 5% (w/v) ovalbumine and 1% (v/v) Tween 80. After washing with PBS/Tween 80, the arrays were incubated with HRP-labeled goat anti-bovine IgG (1/1000 dilution) for 1 hour at 25° C. Following another wash, 2,2′-azino-di-3-ethylbenzthiazoline sulfonate (ABTS) and 20 μl/ml of 3% H2O2 was added to the arrays. One hour after the incubation, colour development was measured and quantified using a charge coupled device (CCD)—camera and image processing system as described by Slootstra et al. (1996).

The raw data consists of optical values that were obtained by the CCD-camera, which was used to capture a picture of the card before and after conducting the peroxidase colouring. Both pictures were then subtracted from each other, resulting in a value for each specific peptide. The values of wells containing air bubbles, which may cause false-positive values, were scored as zero. A separate set of positive and negative control peptides was synthesised in parallel in order to perform quality verification of the synthesised peptides. These peptides were screened with commercial antibodies 3C9 and 57.9 (Posthumus et al., 1990).

Further data analysis was performed with the creation of boxplots, linear intensity profiles and heat maps. In order to classify a specific peptide as a potential protection sites, a TukeyHSD test was performed on the raw data set. Potential protection sites were only selected if the peptide recognition by serum from the native vaccine group was significantly higher than both P. pastoris vaccine group and the negative control group.

An indirect ELISA was used to re-evaluate the recognition of the peptides, that were classified as potential protection sites, by pooled and individual serum samples from animals vaccinated with either native Oo-ASP, P. pastoris Oo-ASP or QuilA adjuvant. To sustain linear test results, serum samples for the ELISAs were taken from the same animals as the ones used for the peptide microarray analysis. 96-well ELISA plates (MaxiSorp, NUNC) were coated with 0.25 pg/ml peptide solution in 100 μl coating buffer (0.05 M Carbonate-Bicarbonate buffer pH 9.6) for 20 hours at 4° C. The plates were sequentially blocked with 200 μl blocking buffer (2% BSA in 150 mM PBS/Tween 20) for 1 hour at room temperature, whereas the in-between wash steps were conducted with 300 μl wash buffer (150 mM PBS/Tween20). Next, the plates were incubated with pooled or individual bovine serum samples (1/200 dilution in PBS) at room temperature for 1 hour. After another wash step, the plates were incubated with HRP-labeled sheep anti-bovine IgG (1/1000 dilution in blocking buffer) at room temperature for 1 hour. After a last wash step, ABTS was added to the plates, which were incubated for 10 minutes at room temperature. The colour development due to oxidation of ABTS, expressed as OD405-492, was quantified by using an Infinite F50 Absorbance Microplate Reader (Tecan Trading AG; Männedorf, Switzerland).

In addition, a competitive inhibition ELISA was performed to evaluate if the peptides are able to inhibit the binding of native Oo-ASP by serum antibodies from animals vaccinated with native Oo-ASP. For this, 96-well ELISA plates were coated with 1 μg/ml Oo-ASP in 100 μl coating buffer for hours at 4° C. The plates were sequentially blocked with 150 μl blocking buffer for 1 hour at room temperature, whereas the in-between wash steps were conducted with 200 μl wash buffer. Simultaneously, serum from vaccinated animals was pre-incubated with either native Oo-ASP or one of the peptides at a concentration range of 0-500 pmol/ml for 1 hour at room temperature and then transferred onto the coated ELISA plates for an additional 1-hour incubation. Finally, HRP-labeled sheep anti-bovine IgG1 (1/1000 dilution in blocking buffer) was added to the plates and after a 1-hour incubation, supplemented with ABTS. An Infinite F50 Absorbance Microplate Reader was employed to determine the OD405-492.

### Glycan Microarray

Leading up to the glycan microarrays, ASP-specific antibodies were isolated in order to prevent false positives and a high background fluorescence due to glycan recognition by non-ASP-specific antibodies. Bovine serum samples were obtained from animals vaccinated with either endogenous Oo-ASP or P. pastoris Oo-ASP, one week after the third vaccination. In addition, serum samples from cattle vaccinated only with the QuilA adjuvant were acquired to serve as negative control in the in vitro studies described below. In order to obtain ASP-specific antibodies, lipoproteins and lipids were first removed from serum as they can potentially clog the purification columns. This was carried out through the addition of a 10% dextran sulphate and a 1M calcium chloride solution, followed by centrifugation at 10,000×g. Due to the small volume of these samples, Protein G HP SpinTrap columns (GE Life Sciences) were utilised to initially purify IgG antibodies, according to the manufacturer's protocol. The IgG's were subsequently brought onto NHS HP SpinTrap colums (GE Life Sciences) coated with endogenous Oo-ASP antigens, according to the manufacturer's protocol. Buffers were always adapted to suit the requirements of both SpinTrap columns. On that account, only IgG's that bind to the endogenous antigens were purified from the different serum samples. In other words, antibodies induced by P. pastoris Oo-ASP that do not possess any level of cross reactivity towards their endogenous counterpart were not captured. After purification, all samples were dialyzed repeatedly with Slide-A-Lyzer MINI dialysis devices (ThermoFischer) in order to obtain a PBS-buffered solution. Thereafter, all samples were concentrated using Amicon Ultra-15 Centrifugal Filter Units (Merck Millipore) and antibody activity was evaluated through western blot (data not shown).

Synthetic glycan microarray slides were thawed at room temperature and subsequently covered with a silicone gasket for the formation of wells, each on top of one array of synthetically printed N-glycans. At first, the arrays were incubated with 300 μl of ASP-specific antibodies (1/500 in PBS-0.01% Tween20 and 1% BSA) from either native Oo-ASP or P. pastoris Oo-ASP vaccinated animals. Serum from QuilA vaccinated animals, also purified for ASP specificity, was used as negative control. Additionally, one well served as a negative technical control and did not contain antibodies. After a one-hour incubation at room temperature whilst shaking, the slides were washed with PBS-0.05% Tween20 and PBS. For the incubation with secondary antibodies, the wells were filled with a solution containing Cy3-labeled anti-bovine IgG (1/1000 dilution in PBS-0.01% Tween20 and 1% BSA) and kept in the dark at room temperature for 30 minutes, whilst shaking. Finally, the slides were sequentially washed with PBS-0.05% Tween 20, PBS and deionized water, before being dried and kept in the dark until further use. The binding of N-glycans by ASP-specific antibodies was then determined using a G2565BA scanner (Agilent Technologies, CA, USA) with a 532 nm laser. Fluorescently-labelled bovine serum albumin (BSA) was included as an array printing control and always composes the first four spots on each glycan microarray.

The data from the G2565BA scanner was analysed through GenePix Pro 7.0 software (Molecular Devices, CA, USA) by implementing a spot-finding algorithm that was used to align and re-size fluorescence spots in the microarray images. The median fluorescence intensity (MFI) for each of the four spots was then obtained and exported to Microsoft Excel software, where the background MFI was subtracted for each of the spots. For further analysis, the four technical replicates per glycan structure were exported to GraphPad Prism (version 6.0c, Fay Avenue, La Jolla, CA, USA) for statistical analysis and data visualisation. As each array contained 135 structures in quadruplicate, a P-value of 0.001 was adopted to minimize the chance of erroneous rejection of the HO.

### Results

### 1.Peptide Microarray

The analysis performed by PEPSCAN Presto BV did not reveal a significant difference in peptide recognition between the three different vaccine groups. One out of five serum samples from native Oo-ASP vaccinated cattle was able to recognize peptides which were not recognized by P. pastoris Oo-ASP and control vaccine groups. In addition, a TukeyHSD analysis was performed on the raw data set and only the samples that had a p<0.05 for both native vs. recombinant and native vs. control group were selected as potential protection sites. Based on this analysis, four peptides (underlined in FIG. 1) were selected for further evaluating by immunological in vitro assays.

### 2.ELISA

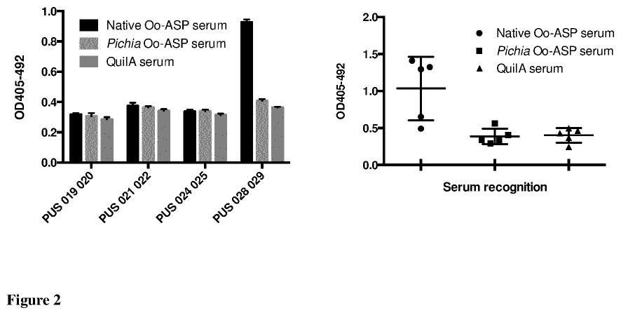

The indirect ELISA with the four selected peptides demonstrated that one of the four peptides, PUS 028 029, was strongly recognized by both pooled and individual serum samples from native Oo-ASP vaccinated animals, whilst this recognition is substantially lower for P. pastoris Oo-ASP and QuilA control vaccine groups (FIG. 2).

The inhibition ELISA demonstrated that none of the selected peptides was able to inhibit the binding of native Oo-ASP induced antibodies to native Oo-ASP coated on the ELISA plate. Also, a mixture of the four peptides did not have any inhibitory capacity.

### 3.Glycan Microarray

The glycan microarray results, projected in FIGS. 3A & 3B, demonstrate the recognition of a varied battery of N-glycans by native Oo-ASP specific IgG's. With exceptions, all recognised N-glycans contain a core α1,3-fucose, optionally in combination with N-glycans containing a α1,6-fucose. There were no N-glycans with core α1,3-fucose that were not recognised by these antigen-specific antibodies. Recognition of the N-glycans on this array by P. pastoris induced native Oo-ASP specific IgG's was mostly absent, with the exception of the trimannosidic structure G99 (data now shown).

### Example 3

### Recombinant Expression of Fucosylated ASP Antigens

Expression of recombinant ASP antigens in Nicotiana benthamiana is achieved by the infiltration of Agrobacterium tumefaciens, a natural pathogen of plants that is able to transfer pieces of DNA into the genome of the plant. Engineered Agrobacterium clones (strain MOG101) harbor pHYG expression plasmids with in-house codon-optimized genes encoding the mature ASP antigen. Expression from these plasmids is driven by a dual 35S promoter and protein yield is further enhanced by co-infiltration of a viral silencing suppressor. In order to glyco-engineer the recombinant ASP antigens, an Agrobacterium clone harboring a construct that drives the expression of a core modifying fucosyltransferase can be added to the infiltration mix. Fucosyltransferases that have been used to successfully modify the N-glycan core are fucosyltransferase 8 from mouse or fruit fly in the case of core alpha-1,6-fucose, and fucosyltransferase C from Schistosoma mansoni or plant endogenous fucosyltransferase 11 or 12 in the case of core alpha-1,3-fucose.

Typically, leaves of 5- to 6-week-old ΔXT/FT plants (RNAi knockdown plants that lack endogenous core alpha-1,3-fucose and beta-1,2-xylose) are infiltrated with multiple Agrobacterium clones, which facilitates transient protein production and simultaneous glyco-engineering of ASP antigens in 5-6 days. At this stage, infiltrated leaves are harvested and vacuum-infiltrated with extraction buffer (50 mM sodium acetate buffer at pH 4.4) to isolate recombinant ASP antigen from the extracellular space (apoplast). The recombinant ASP antigens are then purified from the collected apoplast fluid using HS Poros 50 cation exchange chromatography at pH 4.4.

In order to evaluate the ability of core alpha 1,6 and core alpha 1,3 fucosylated recombinant N. benthamiana ASP antigens to inhibit the binding of anti-ASP antibodies to native ASP, a competition ELISA was conducted. For this, the wells of an ELISA-maxisorp plate were coated with 100 μl of 0.5 pg/ml native Oo-ASP in 0.05M carbonate coating buffer (pH 9.6) and incubated overnight at 4° C. After a 150 mM PBST_{0.05} wash, which were conducted three times between each step, all wells were blocked with 200 μl of 2% bovine serum albumin in 150 mM PBST_{0.05} for one hour at room temperature. During the blocking, a separate low-binding 96-well plate was used to pre-incubate pooled serum from native Oo-ASP vaccinated animals with the different recombinant antigens, which were added at different concentrations ranging from 0-1000 pmol/ml. After blocking, the content of the pre-incubation plate was added onto the ELISA-maxisorp plate in order to bring the native and recombinant antigens into competition for anti-ASP antibody binding. After another incubation for 1 hour at room temperature, 100 μl goat anti-bovine IgG1-HRP was added at a 1/500 dilution and was incubated for 1 hour at room temperature. The development of the ELISA was then conducted with ABTS (Roche) and optical density at 405 nm was measured per well and corrected by the optical density at 492 nm for background.

### Results

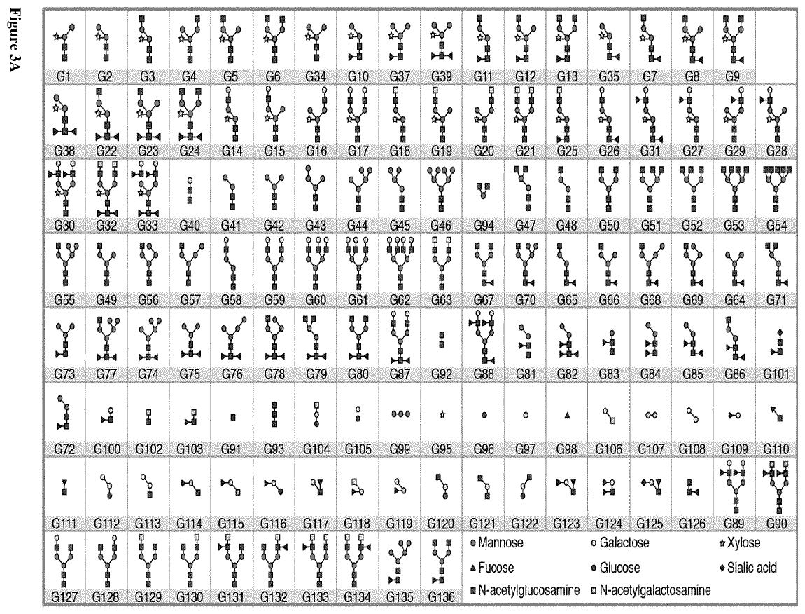

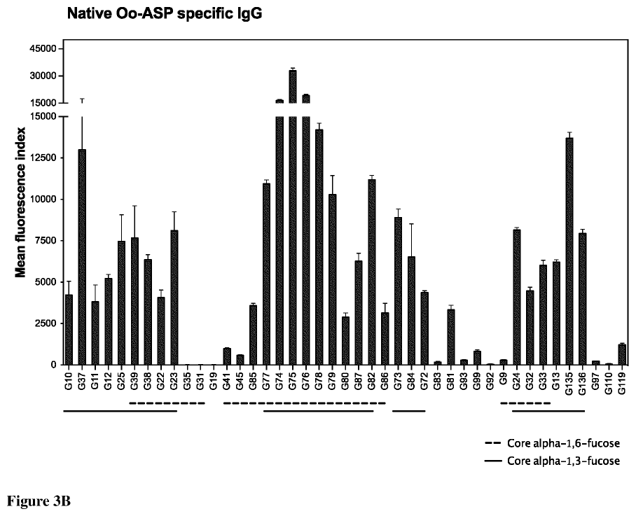

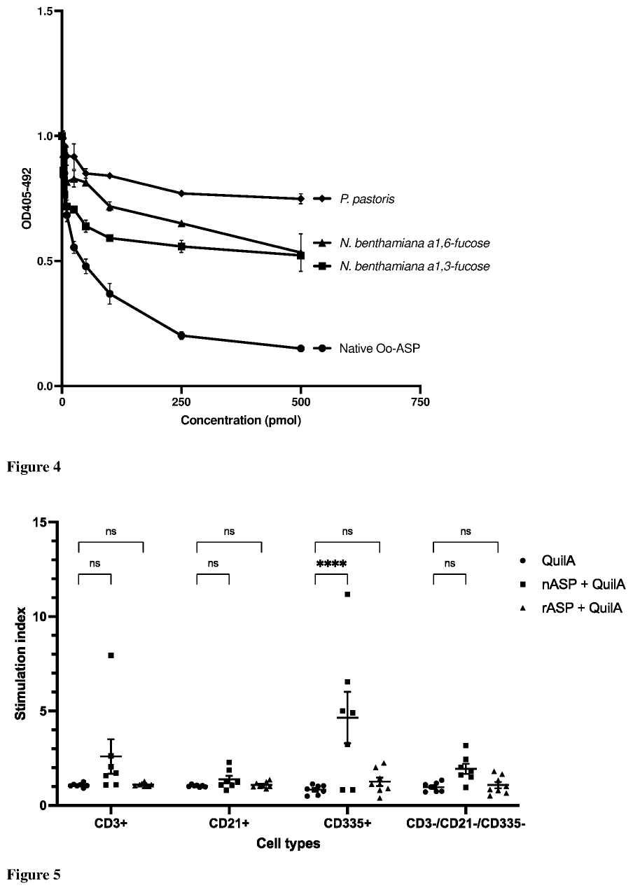

The results in FIG. 4 demonstrate that the N. benthamiana recombinant ASPs are able to compete with the native Oo-ASP for anti-ASP antibody binding, whilst this is much less the case for the P. pastoris recombinant ASP. These results indicate a higher antibody affinity towards the N. benthamiana recombinants if compared to the P. pastoris. These findings can be explained by the presence of specific antibody epitopes present on both the native Oo-ASP and the N. benthamiana recombinant ASPs, which are not present on the P. pastoris. Most likely, these high affinity antibody epitopes (partly) comprise the N-glycosylation as this is the main difference between the N. benthamiana recombinants and the P. pastoris recombinant, specifically concerning core fucosylation. At lower concentrations, a comparison of N. benthamiana recombinants containing either a core α-1,3-fucose or core α-1,6-fucose indicates that the core α-1,3-fucose containing recombinant is able to inhibit the binding of antibodies to the native antigen slightly better than its core α-1,6-fucose counterpart. This may indicate the presence of some highly specific antibody epitopes that are shared with the native antigen.

### Example 4

For evaluating the N. benthamiana recombinant ASPs in cattle, twenty-four cows were randomly divided into three vaccine groups of eight animals. Alongside the rASP+QuilA vaccine group, a native ASP+QuilA and a negative control group with solely QuilA were included in the trial. Negative control animals received 750 pg QuilA per vaccination, whereas the recombinant and native vaccine groups received 30 μg of the respective antigens in combination with 750 pg QuilA. The recombinant vaccine group received a 50/50 ratio of antigens with either core α-1,3-fucose or core α-1,6-fucose containing N-glycans. Prior to the study, all animals were confirmed negative for recent O. ostertagi infections through performing fecal egg counts and ELISA. The cows were immunized three times with a three-week interval and were exposed to a trickle infection of 1000 L3/day for 25 days starting on the day of the third immunization.

In order to evaluate both the humoral and cellular response, blood was collected one week after each immunization and at necropsy. Peripheral blood mononuclear cells (PBMCs) were isolated from blood samples through Lymphoprep gradient centrifugation. In addition, mononuclear cells (MNC) from draining abomasal lymph nodes were obtained through mechanical disruption of the tissue through a 70 μm cell strainer, followed by Lymphoprep gradient centrifugation. These cells were labelled with PKH26 and cultured at 2.5×10⁵ cells/200 μl RPMI complete medium with either medium alone or medium with 5 μg/ml nASP for 5 days. To obtain the PKH26 starting intensity, a small portion of the PKH26 labelled cells was used and stained with the following antibody mix: CD3-IgG1, CD21-IgM, CD335-IgG2b, IgG1-V450, IgM-APC-Cy7, IgG2b-FITC and a viability dye. After 5 days of culture, cells were harvested and stained with the antibody mix described earlier in order to obtain the final PKH26 intensity per cell type. ModFit LT software was then used to calculate the proliferation index for each cell population. Stimulation index was achieved by dividing the nASP stimulated cells by the medium stimulated cells, essentially representing the fold change. Enzyme-linked immunosorbent assays (ELISAs) were performed to study both the systemic and local (abomasal) (cross)reactive IgG1, IgG2 and IgA response in the three vaccine groups. For this, nASP was coated in 96-well Maxisorp plates at 1 μg/ml in carbonate buffer (pH 9.6) overnight. Wells were then blocked with 2% BSA in PBST_{0.05} and wash steps were conducted in threefold with PBST_{0.05}. After blocking, wells were incubated with the bovine serum samples at a dilution of 1/200 for 1 hour. HRP-conjugated anti-bovine IgG1, IgG2 and IgA were added to the wells for an incubation of 1 hour at room temperature, before the addition of 2,2′-azinobis (3-ethylbenzthiazoline-6-sulfonic acid) (ABTS) substrate in order to develop the ELISA. The optical density was measured at 405 nm. Parasitological parameters were evaluated at described previously in Van Meulder et al. (2015) and Vlaminck et al. (2015). In short, fecal egg counts were conducted three times per week with the McMaster method at a sensitivity of 25 eggs per gram feces (EPG), starting 21 days after the first infection until necropsy. Adult worms for worm counting and worm length measurements were obtained by washing the abomasum after necropsy, whereas L4 were collected after performing a digestion of the abomasal mucosae with hydrochloric acid-pepsin.

### Results

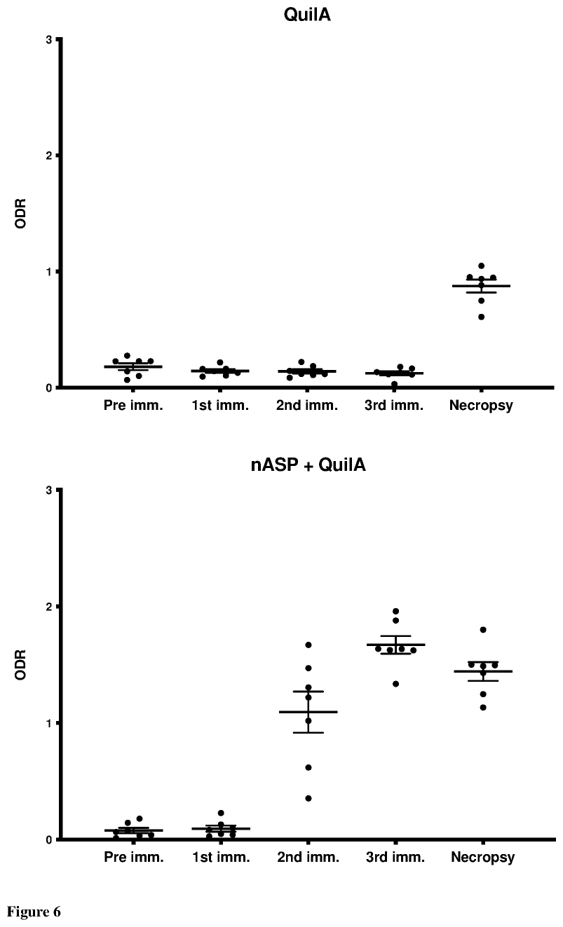

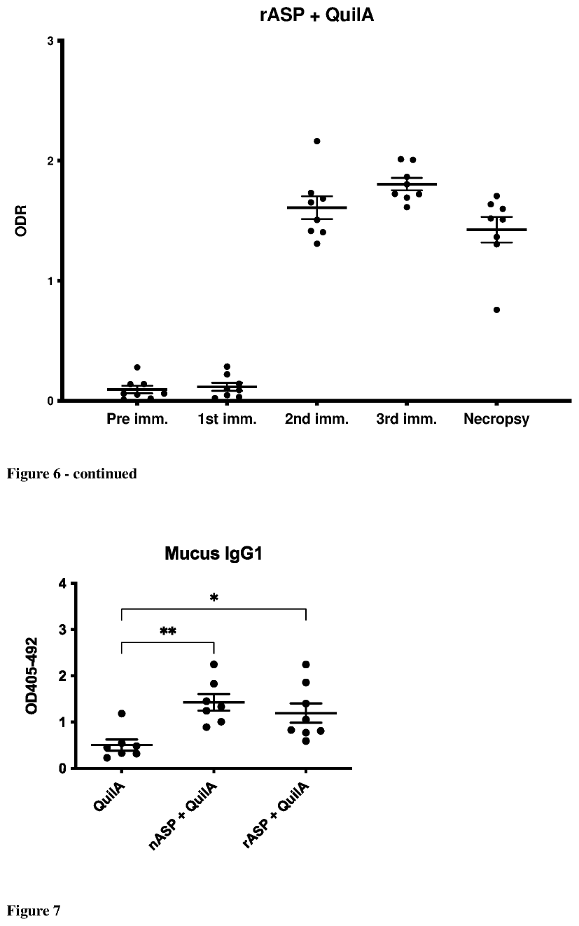

PBMC cell proliferation upon (re-)exposure to the nASP antigen was mostly present in the nASP vaccine group, specifically for CD335+ NK cells (FIG. 5). This was not or less the case for the QuilA controls and rASP immunized cows. MNC cell proliferation upon antigen (re-)exposure was limited and less conclusive (data not shown). The ELISA results evaluating the systemic antibody response demonstrates mostly an IgG1 response with (cross)reactive antibodies that recognize nASP (FIG. 6). For the two vaccine groups with either the nASP or rASP antigens included, the IgG1 response was seen 1 week after the second immunization and onwards. A more limited IgG2 response was present and follows a similar pattern as described for IgG1 (data not shown). Remarkably, a clear local IgG1 and IgG2 response was described in the nASP and rASP groups (FIG. 7), whilst this was not or less the case for control animals and also not seen with previous recombinant antigens produced in P. pastoris.

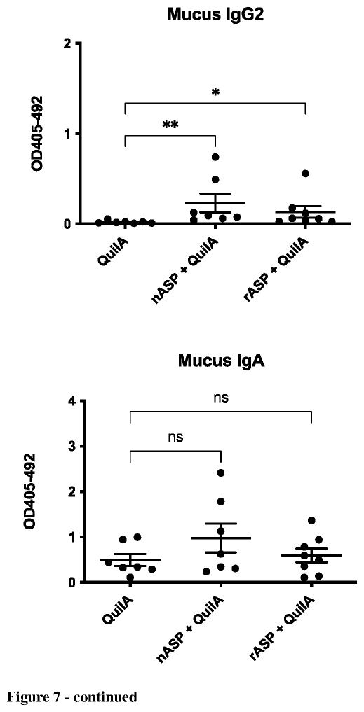

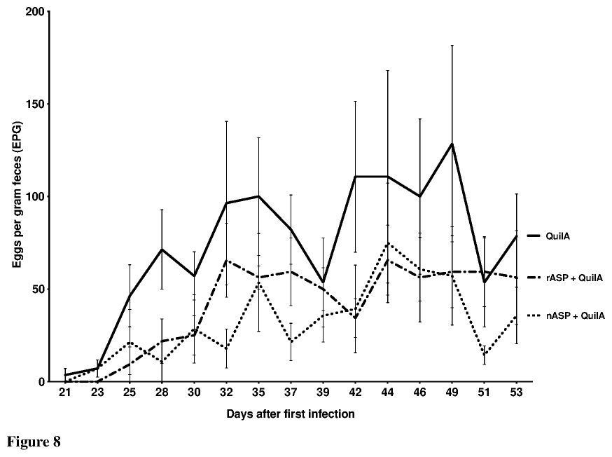

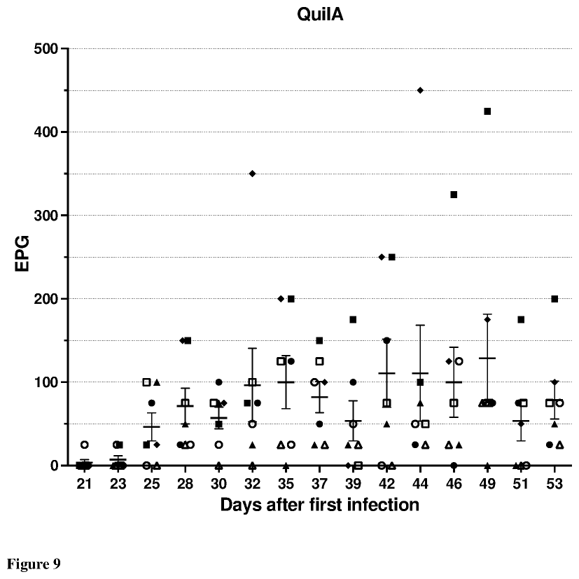

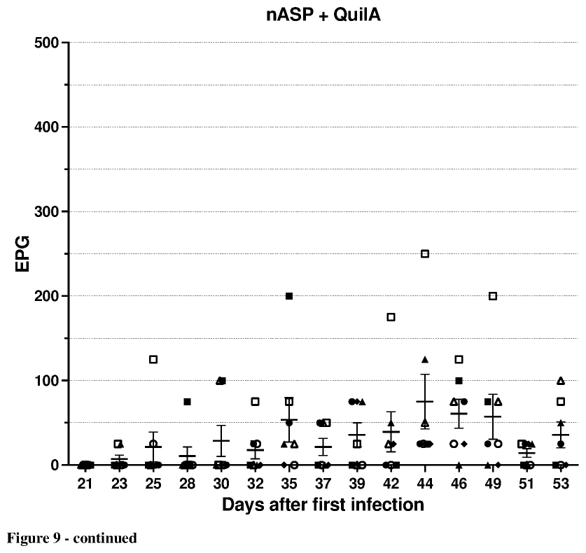

Fecal egg counts demonstrated in FIG. 8 indicate an overall reduction in both the nASP and rASP vaccine groups, compared to the QuilA controls. Although animals vaccinated with nASP or rASP still excrete O. ostertagi eggs, the egg excretion appears to be delayed and importantly both antigens prevent animals from excreting large amounts of eggs, the latter demonstrated in FIG. 9. Last, the cumulative egg counts were obtained by calculating the area under the curve (AUC) per animal (FIG. 10 panel A). Compared to the QuilA controls, a significant reduction of 57% was observed for animals immunized with nASP+QuilA, whereas a similar non-significant reduction of 45% was observed for animals immunized with rASP+QuilA. Based on these result, a second vaccination study was performed with the rASP. In comparison to the QuilA controls, the rASP+QuilA immunized animals showed a significant reduction of 39% in cumulative egg counts (FIG. 10 panel B).

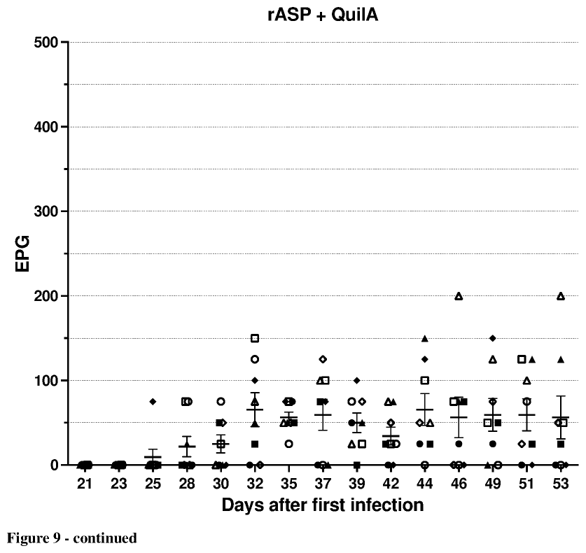

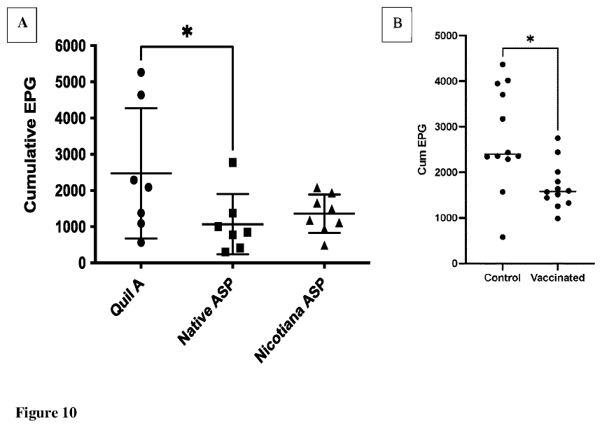

Concerning worm counts, no clear differences in total worms, total adults or total L4 were found in the first study if comparing the QuilA controls to the nASP and rASP vaccine groups (FIG. 11). However, if comparing ratio of total L4/total worms per vaccine group, an increase was observed in the nASP vaccine group compared to the other groups, however this was only the case for 2 out of 7 animals. No clear differences in worm length were described between the three vaccine groups in study 1 (FIG. 12).

### Example 5

### Obtaining Endogenous and Recombinant ASP

Collection of adult C. oncophora worms, preparation of the excretion/secretion material and purification of the dd-ASP protein fraction were carried out as described previously (Borloo et al., 2013). Recombinant version of the Cooperia ASP was expressed in P. pastoris and purified as described in Gonzalez-Hernández et al. (2018).

### Glycan Microarray

Leading up to the glycan microarrays for Cooperia oncophora, ASP-specific antibodies were isolated in order to prevent false positives and a high background fluorescence due to glycan recognition by non-ASP-specific antibodies. Bovine serum samples were obtained from animals vaccinated with either endogenous Co-dd-ASP or P. pastoris Co-dd-ASP, one week after the third vaccination. Serum samples from cattle vaccinated only with the QuilA adjuvant were acquired to serve as negative control in the in vitro studies described below. In order to obtain ASP-specific antibodies, lipoproteins and lipids were first removed from serum as they can potentially clog the purification columns. This was carried out through the addition of a 10% dextran sulphate and a 1M calcium chloride solution, followed by centrifugation at 10,000×g. Due to the small volume of these samples, Protein G HP SpinTrap columns (GE Life Sciences) were utilized to initially purify immunoglobulins (Ig's), according to the manufacturer's protocol. The immunoglobulins were subsequently brought onto NHS HP SpinTrap colums (GE Life Sciences) coated with endogenous Co-dd-ASP antigens, according to the manufacturer's protocol. Buffers were always adapted to suit the requirements of both SpinTrap columns. Only antibodies that bind to the endogenous antigens were purified from the different serum samples. Therefore, antibodies induced by P. pastoris Co-dd-ASP that do not possess any level of cross reactivity towards their endogenous counterpart were not captured. After purification, all samples were dialyzed repeatedly with Slide-A-Lyzer MINI dialysis devices (ThermoFischer) in order to obtain a PBS-buffered solution. Thereafter, all samples were concentrated using an Amicon Ultra-15 Centrifugal Filter Unit (Merck Millipore) and antibody activity was evaluated through western blot (data not shown).

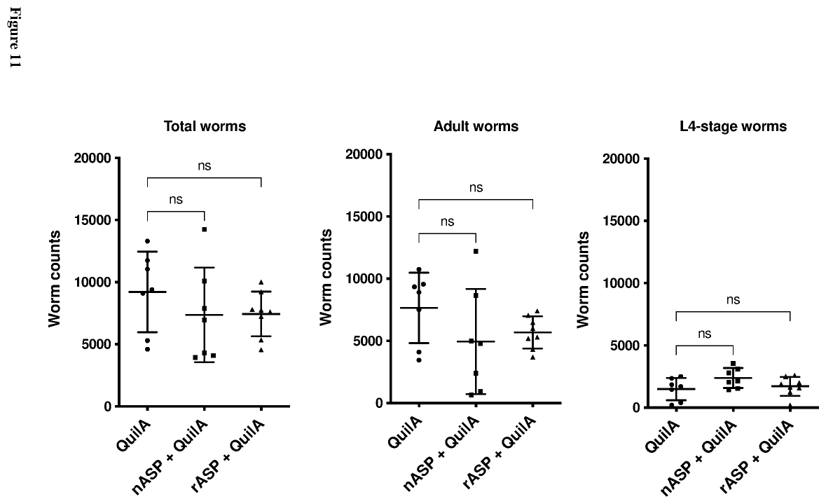

Synthetic microarray slides (FIG. 13) were thawed at room temperature and subsequently covered with a silicone gasket for the formation of wells, each on top of one array of synthetically printed N-glycans. At first, the arrays were incubated with 300 μl of ASP-specific antibodies (1/500 in PBS-0.01% Tween20 and 1% BSA) from either native Co-dd-ASP or P. pastoris Co-dd-ASP vaccinated animals. Serum from QuilA vaccinated animals, also purified for ASP specificity, was used as negative control. Additionally, one well served as a negative technical control and did not contain antibodies. After one hour incubation at room temperature whilst shaking, the slides were washed with PBS-0.05% Tween20 and PBS. For the incubation with secondary antibodies, the wells were filled with a solution containing Cy3-labeled anti-bovine IgG (1/1000 dilution in PBS-0.01% Tween20 and 1% BSA) and kept in the dark at room temperature for 30 minutes, whilst shaking. Finally, the slides were sequentially washed with PBS-0.05% Tween 20, PBS and deionized water, before being dried and kept in the dark until further use. The binding of N-glycans by ASP-specific antibodies was then determined using a G2565BA scanner (Agilent Technologies, CA, USA) with a 532 nm laser. Fluorescently-labelled bovine serum albumin (BSA) was included as an array printing control.

The data from the G2565BA scanner was analysed through GenePix Pro 7.0 software (Molecular Devices, CA, USA) by implementing a spot-finding algorithm that was used to align and re-size fluorescence spots in the microarray images. The median fluorescence intensity (MFI) for each of the four spots was then obtained and exported to Microsoft Excel software, where the background MFI was subtracted for each of the spots. For further analysis, the four technical replicates per glycan structure were exported to GraphPad Prism (version 6.0c, Fay Avenue, La Jolla, CA, USA) for statistical analysis and data visualisation. As each array contained 135 structures in quadruplicate, a P-value of 0.001 was adopted to minimize the chance of erroneous rejection of the Ho.

### Results

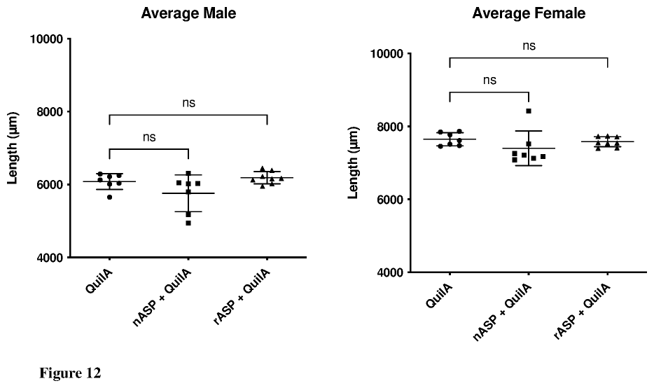

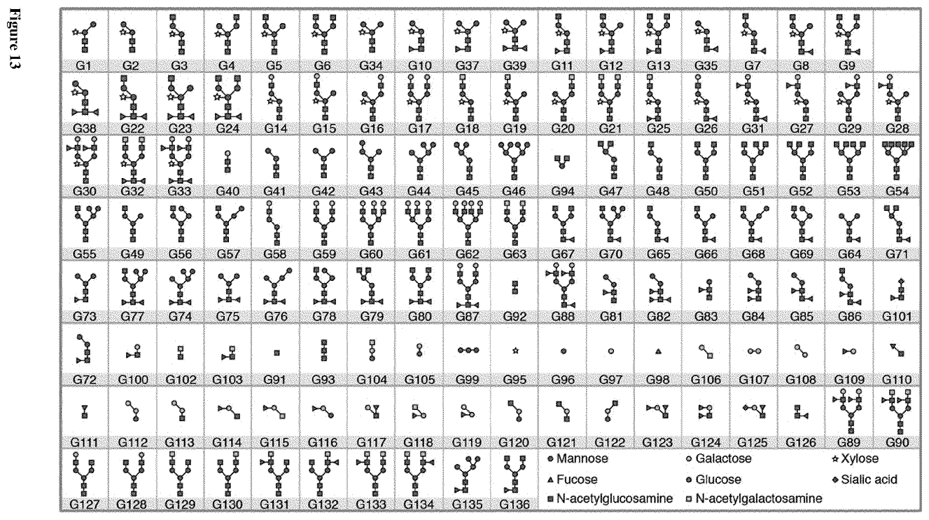

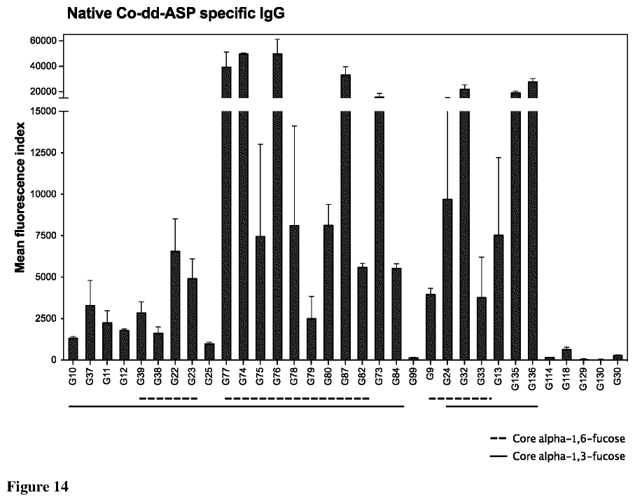

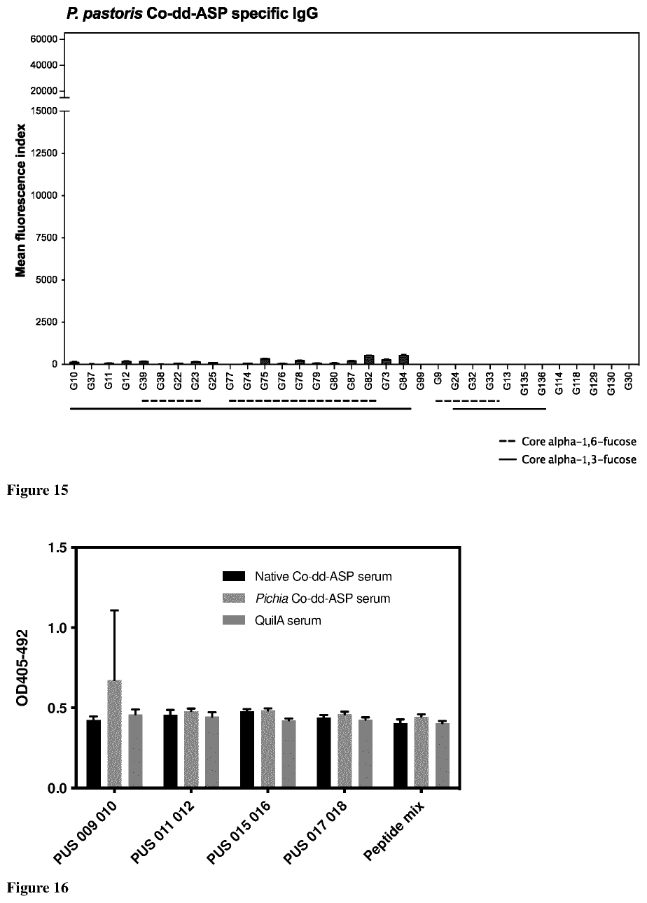

The glycan microarray results, projected in FIG. 14, demonstrate the recognition of a varied battery of N-glycans by native Co-dd-ASP specific antibodies. With exceptions, all recognised N-glycans contain either a core α1,3-fucose, α1,6-fucose or combination thereof. There were no N-glycans with core α1,3-fucose that were not recognised by these antibodies. Recognition of the N-glycans on this array by native ASP-specific antibodies from P. pastoris ASP vaccinated animals was substantially lower compared to antibodies from native Co-dd-ASP vaccinated animals (FIG. 15).

### Example 6

A peptide microarray was carried out by Pepscan Presto BV (Netherlands) in order to obtain insights in the recognition of Cooperia oncophora protein epitopes by protective IgG antibodies. An array of linear peptide epitopes was generated by splitting the amino acid sequence of Co-dd-ASP in overlapping fragments in silico on a solid support called “mini-card”. Additionally, conformational epitopes were constructed through the Chemically Linked Peptides on Scaffolds (CLIPS) technology (Timmerman et al., 2007), also in overlapping fragments, which allows mimicking of secondary structure elements, such as loops, α-helixes and β-strands. Constructs that form an incomplete part of the antibody epitope may be recognised by antibodies, albeit with a lower affinity, whereas constructs that are not a part of this epitope are not recognised. The overlapping battery of ASP peptides was synthesized using solid-phase 9-fluorenylmethoxycarbonyl (Fmoc) synthesis and an amino functionalised polypropylene support was obtained by grafting with a proprietary hydrophilic polymer formulation. This was followed by a reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups through the addition of trifluoroacetic acid (TFA). A standard Fmoc-peptide synthesis was used to synthesize peptides on the amino-functionalised solid support.

The binding of serum antibodies to each peptide was evaluated in a pepscan-based ELISA. This was conducted with serum samples from cattle vaccinated with either native Co-dd-ASP (n=5), P. pastoris Co-dd-ASP (n=5) or only QuilA adjuvants as control (n=5), which were obtained one week after the third immunisation. In order to prevent deviations in antibody binding, all pre-coat conditions (antibody concentration and the relative amount of competing proteins in the ELISA buffer) were equalised between the different serum samples. At first, array cards with the peptide arrays were incubated overnight at 4° C. with the serum antibodies in PBS containing 5% (v/v) horse serum, 5% (w/v) ovalbumine and 1% (v/v) Tween 80. After washing with PBS/Tween 80, the arrays were incubated with HRP-labeled goat anti-bovine IgG (1/1000 dilution) for 1 hour at 25° C. Following another wash, 2,2′-azino-di-3-ethylbenzthiazoline sulfonate (ABTS) and 20 μl/ml of 3% H₂O₂ was added to the arrays. One hour after the incubation, colour development was measured and quantified using a charge coupled device (CCD)—camera and image processing system as described by Slootstra et al. (1996).

The raw data consists of optical values that were obtained by the CCD-camera, which was used to capture a picture of the card before and after conducting the peroxidase colouring. Both pictures were then subtracted from each other, resulting in a value for each specific peptide. The values of wells containing air bubbles, which may cause false-positive values, were scored as zero. A separate set of positive and negative control peptides was synthesised in parallel in order to perform quality verification of the synthesised peptides. These peptides were screened with commercial antibodies 3C9 and 57.9 (Posthumus et al., 1990 J. Virol. 64).

Further data analysis was performed with the creation of boxplots, linear intensity profiles and heat maps. In order to classify a specific peptide as a ‘protective’ antibody epitope, a TukeyHSD test was performed on the raw data set. Potential ‘protective’ antibody epitopes were only selected if the peptide recognition by serum from the native vaccine group was significantly higher than both P. pastoris vaccine group and the negative control group.

An indirect ELISA was used to re-evaluate the recognition of the peptides, that were classified as potential ‘protective’ epitopes, by pooled samples from animals vaccinated with either native Co-dd-ASP, P. pastoris Co-dd-ASP or QuilA adjuvant. To sustain linear test results, serum samples for the ELISAs were taken from the same animals as the ones used for the peptide microarray analysis. 96-well ELISA plates (MaxiSorp, NUNC) were coated with 0.25 pg/ml peptide solution in 100 μl coating buffer (0.05 M Carbonate-Bicarbonate buffer pH 9.6) for 20 hours at 4° C. The plates were sequentially blocked with 200 μl blocking buffer (2% BSA in 150 mM PBS/Tween 20) for 1 hour at room temperature, whereas the in-between wash steps were conducted with 300 μl wash buffer (150 mM PBS/Tween20). Next, the plates were incubated with pooled or individual bovine serum samples (1/200 dilution in PBS) at room temperature for 1 hour. After another wash step, the plates were incubated with HRP-labeled sheep anti-bovine IgG (1/1000 dilution in blocking buffer) at room temperature for 1 hour. After a last wash step, ABTS was added to the plates, which were incubated for 10 minutes at room temperature. The colour development due to oxidation of ABTS, expressed as OD405-492, was quantified by using an Infinite F50 Absorbance Microplate Reader (Tecan Trading AG; Männedorf, Switzerland).

In addition, a competitive inhibition ELISA was performed to evaluate if the peptides are able to inhibit the binding of native Co-dd-ASP by serum antibodies from animals vaccinated with native Co-dd-ASP. For this, 96-well ELISA plates were coated with 1 μg/ml Co-dd-ASP in 100 μl coating buffer for 20 hours at 4° C. The plates were sequentially blocked with 150 μl blocking buffer for 1 hour at room temperature, whereas the in-between wash steps were conducted with 200 μl wash buffer. Simultaneously, serum from vaccinated animals was pre-incubated with either native Co-dd-ASP or one of the peptides at a concentration range of 0-500 pmol/ml for 1 hour at room temperature and then transferred onto the coated ELISA plates for an additional 1-hour incubation. Finally, HRP-labeled sheep anti-bovine IgG1 (1/1000 dilution in blocking buffer) was added to the plates and after a 1-hour incubation, supplemented with ABTS. An Infinite F50 Absorbance Microplate Reader was employed to determine the OD405-492.

### Results

A TukeyHSD analysis was performed on the raw data set and only the peptides that had a p <0.05 for both native Co-dd-ASP vs. recombinant Co-dd-ASP and native Co-dd-ASP vs. control group were selected as potential ‘protective’ antibody epitope. Based on this analysis, four peptides were selected for further evaluation by immunological in vitro assays. The indirect ELISA with the selected peptides demonstrated that there was no clear difference in peptide recognition by both pooled serum samples from either native Co-dd-ASP, P. pastoris Co-dd-ASP or QuilA vaccinated animals (FIG. 16). In addition, an inhibition ELISA demonstrated that none of the selected peptides, or a mixture thereof, was able to inhibit the binding of native Co-dd-ASP induced antibodies to native Co-dd-ASP coated on the ELISA plate.

### Example 7

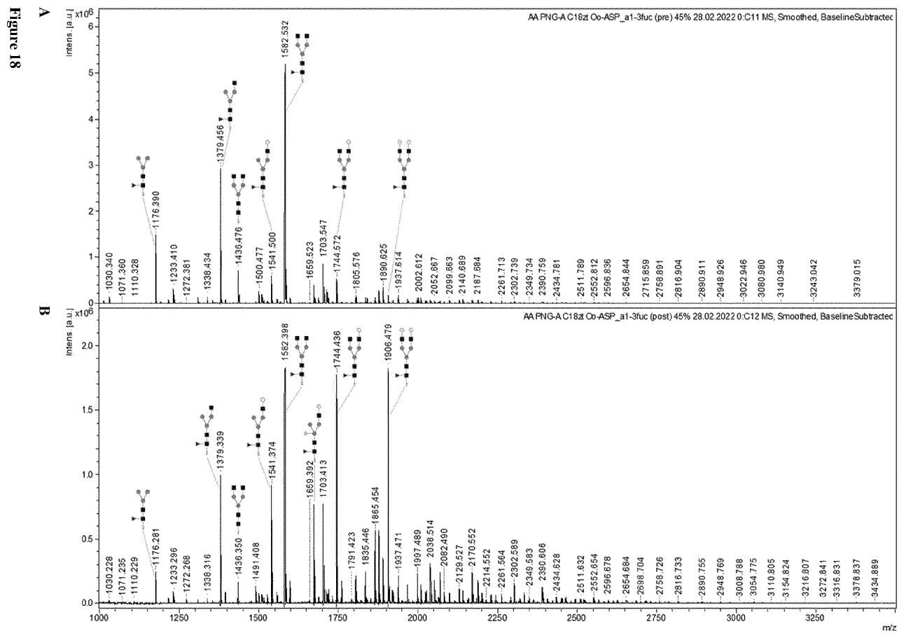

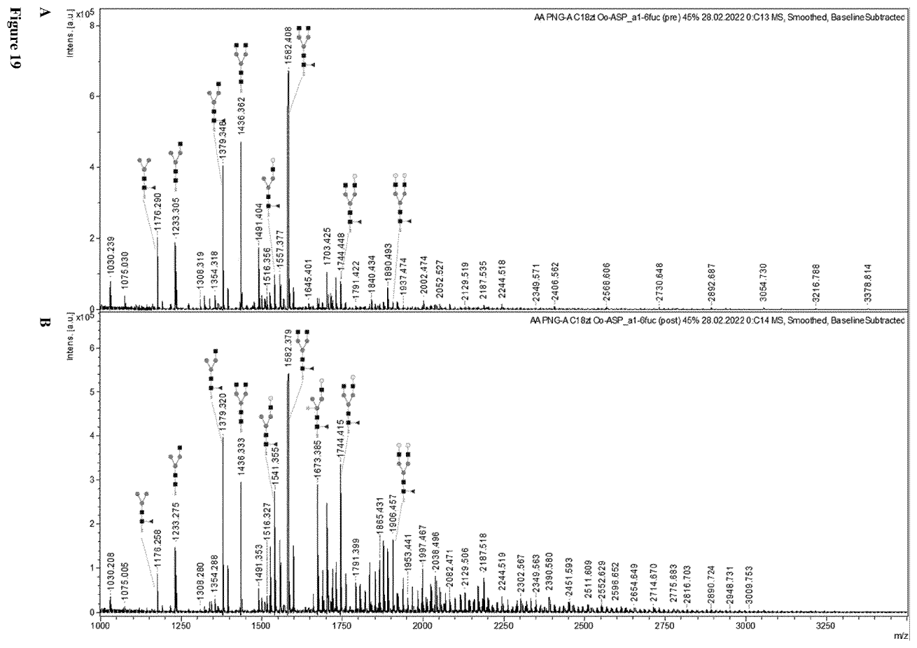

To increase the level of terminal galactose, each glycoform (either with core α1,3-fucose or core α1,6-fucose) of the N. benthamiana Oo-ASP-1 was subjected to affinity chromatography using agarose-bound Ricinus communis agglutinin-I (Vector Laboratories), largely according to the manufacturer's instructions. Adaptations: Pierce Spin Columns (Thermofisher) were filled with 0.5 ml of the RCA I slurry with 5 column volumes of binding/wash buffer, each time centrifuging at 75×g for 1 minute in order to discard the flowthrough. In 0.5 ml binding/wash buffer, N. benthamiana recombinants were applied to the column at a concentration of 1 mg/ml, for an incubation time of 1 hour at 4° C. while constantly mixing by inversion. Elution was done by a two-time application of 0.5 ml of Glycoprotein Eluting Solution (Vector Laboratories), followed by 1 min centrifugation at 100×g. The results are shown in FIGS. 18 and 19.

### Results:

FIG. 18 demonstrates a clear increase in terminally galactosylated N-glycans. N-glycans with terminal galactose also present as the glycoprotein carries two N-glycans. If only one of its N-glycans is galactosylated, it gets purified as a whole.

FIG. 19 also demonstrates a clear increase in terminally galactosylated N-glycans. N-glycans with terminal galactose also present as the glycoprotein carries two N-glycans. If only one of its N-glycans is galactosylated, it gets purified as a whole.

### REFERENCES

Borloo, J. et al. In-Depth Proteomic and Glycomic Analysis of the Adult-Stage Cooperia oncophora Excretome/Secretome. J. Proteome Res. 12(9), 3900-11 (2013a).
Borloo, J. et al. Structure of Ostertagia ostertagi ASP-1: insights into disulfide-mediated cyclization and dimerization. Acta Crystallogr. Sect. D Biol. Crystallogr. 69, 1-11 (2013b).
González-hernández, A. et al. Host protective ASP-based vaccine against the parasitic nematode Ostertagia ostertagi triggers NK cell activation and mixed IgG1-IgG2 response. Scientific Reports 6, 29496 (2016).
Gonzalez-Hernandez et al. (2017) Comparative analysis of the immune responses induced by native and recombinant versions of the ASP-based vaccine against the bovine intestinal parasite Cooperia oncophora. International Journal for Parasitology 2018 January; 48(1):41-49
Ma at al. Protein Glycoengineering: An Approach for Improving Protein Properties, Front. Chem., 23 Jul. 2020, 8, 1-14.
Posthumus W P, Lenstra J A, Schaaper W M, van Nieuwstadt A P, Enjuanes L, Meloen R H. Analysis and simulation of a neutralizing epitope of transmissible gastroenteritis virus. J Virol. 1990 July; 64(7):3304-9.
   Slootstra, J. W., Puijk, W. C., Ligtvoet, G. J., Langeveld, J. P. M. and Meloen, R. H. (1996). Structural aspects of antibody-antigen interaction revealed through small random peptide libraries. Mol. Div. 1, 87-96
Timmerman, P., Puijk, W. C., Meloen, R. H., 2007. Functional reconstruction and synthetic mimicry of a conformational epitope using CLIPS technology. Journal of molecular recognition 20, 283-299.
Van der Kaaij et al., Glyco-Engineering Plants to Produce Helminth Glycoproteins as Prospective Biopharmaceuticals: Recent Advances, Challenges and Future Prospects, Front. Plant Sci., 2022, 13, 1-12.
Van Meulder et al. (2015) Analysis of the protective immune response following intramuscular vaccination of calves against the intestinal parasite Cooperia oncophora. International Journal for Parasitology 2015 45:637-646
Vlaminck, J., Borloo, J., Vercruysse, J., Geldhof, P., Claerebout, E., 2015. Vaccination of calves against Cooperia oncophora with a double-domain activation-associated secreted protein reduces parasite egg output and pasture contamination. Int. J. Parasitol. 45, 209-213.

1. A recombinant activation-associated secreted protein (ASP) or fragment thereof, said ASP or fragment thereof comprising an N-glycan comprising a core α1,3-fucose and/or a core α1,6-fucose, GlcNAc, and Man in the conformation of structure (Ia) or (Ib) 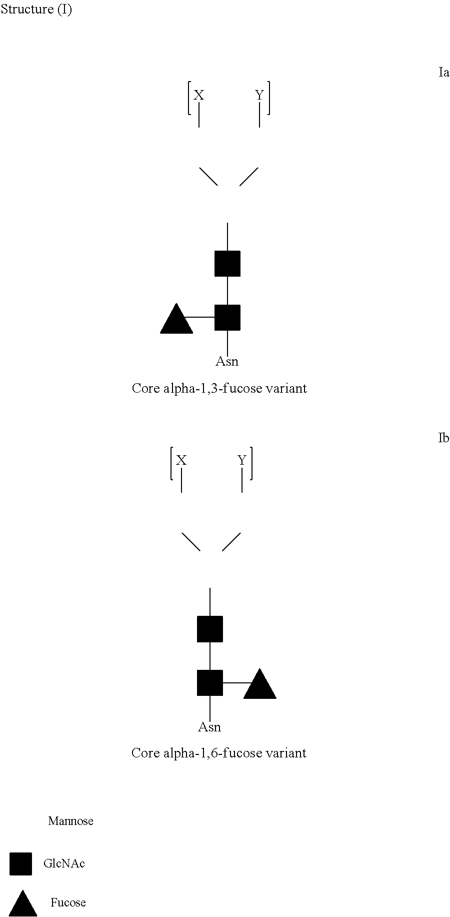
and wherein X and/or Y are absent, or independently from each other substituted with N-acetylglucosamine (GlcNAc), galactose (gal), fucose (fuc), or mannose (man), and combinations thereof.

2. The recombinant ASP or fragment thereof according to claim 1, wherein X and/or Y are substituted with Gal-GlcNAc.

3. The recombinant ASP or fragment thereof according to any one of claim 1 or 2, wherein the recombinant ASP is synthetically produced or obtained from an expression system comprising a fucosyltransferase.

4. The recombinant ASP or fragment thereof according to claim 3, wherein the recombinant ASP or fragment thereof is obtained from an Nicotiana tabacum expression system, Pichia pastoris expression system, or an insect cell line expression system.

5. The recombinant ASP or fragment thereof according to any one of claims 1-4, wherein the amino acid sequence of the recombinant ASP or fragment thereof has at least 90% sequence identity, preferably at least 95% sequence identity, more preferably at least 99%, most preferably 100% sequence identity with the amino acid sequence of ASP of Ostertagia ostertagi, Cooperia oncophora or Teladorsagia circumcincta.

6. The recombinant ASP or fragment thereof according to claim 5, wherein the ASP amino acid sequence of Ostertagia ostertagi is represented by SEQ ID NO: 1, wherein the ASP amino acid sequence of Cooperia oncophora is represented by SEQ ID NOs: 2-4, and wherein the ASP amino acid sequence of Teladorsagia circumcincta is represented by SEQ ID NO: 5.

7. A Pharmaceutical composition comprising the recombinant ASP or fragment thereof according to any one of the claims 1-6 and a pharmaceutically acceptable carrier and/or excipient.

8. A Pharmaceutical composition according to claim 7, wherein the pharmaceutical composition is a vaccine.

9. A Pharmaceutical composition according to claim 8, additionally comprising an adjuvant.

10. The recombinant ASP or fragment thereof according to any one of the claims 1-6 or the pharmaceutical composition according to any one of the claims 7-9, for use as a human or veterinary medicine.

11. The recombinant ASP or fragment thereof according to any one of the claims 1-6, or the pharmaceutical composition according to any one of the claims 7-9, for use in the treatment, prevention and/or reduction of a parasitic nematode infection in a mammal.

12. The recombinant ASP or fragment thereof or the pharmaceutical composition for use according to claim 10 or 11, wherein the parasitic nematode belongs to the genus Ostertagia, and/or to the genus Cooperia and/or to the genus Teladorsagia, in particular wherein the parasitic nematode is Ostertagia ostertagi, Ostertagia leptospicularis, Cooperia oncophora, Cooperia punctate, Cooperia pectinate, Teladorsagia Trifurcata and/or Teladorsagia circumcincta, even more in particular Ostertagia ostertagi, Cooperia oncophora or Teladorsagia circumcincta.

13. A method for producing the recombinant ASP or fragment according to any one of the claims 1-6, said method comprising the steps of:
a) providing an expression system comprising a fucosyltransferase,
b) introducing in the expression system a fucose sugar, in particular a core α1,3-fucose and/or a core α1,6-fucose,
c) expressing the ASP or fragment with the expression system provided in step a) to obtain a recombinant ASP or fragment according to any one of the claims 1-6.

14. A method of treatment, prevention and/or reduction of a parasitic nematode infection in a subject in need thereof, comprising the administration to said subject in need thereof of the recombinant ASP or fragment according to any one of the claims 1-6 or of the pharmaceutical composition according to any one of the claims 7-9.
